(19) United States
(12) Patent Application Publication
(10) Pub. No.: US 2018/0282760 A1
(43) Pub. Date: Oct. 4, 2018

(54) RECOMBINANT MAMMALIAN CELLS AND METHOD FOR PRODUCING SUBSTANCE OF INTEREST

(71) Applicants: TOTO LTD., KITAKYUSHU-SHI (JP); KYUSHU UNIVERSITY, NATIONAL UNIVERSITY CORPORATION, FUKUOKA-SHI (JP)

(72) Inventors: Yoshimasa YAMANA, KITAKYUSHU-SHI (JP); Masako KONDO, KITAKYUSHU-SHI (JP); Masamichi KAMIHIRA, FUKUOKA-SHI (JP)

(21) Appl. No.: 15/941,047

(22) Filed: Mar. 30, 2018

(30) Foreign Application Priority Data
Mar. 31, 2017 (JP) 2017-071936

(51) Int. Cl. C12N 15/85; C12N 9/10; C07K 16/32

(52) U.S. Cl. CPC C12N 15/85; C12N 9/1077; C12Y 204/02008; C12N 2800/30; C07K 2317/10; C07K 2317/24; C07K 16/32

(57) ABSTRACT

There are provided a recombinant cell that enables efficient production of protein, a method for producing the same, and a method for producing protein by using the cell. The cell is a mammalian cell having integrated therein 3 or more expression units containing an exogenous gene of interest, wherein gap elements are provided among multiple expression units, wherein the gap elements are designed to have chain lengths of 0.5 kbp or more, and the gap elements are mutually different sequences. Such a cell can produce recombinant protein stably for a long period and, especially, can show a strong positive linear correlation between the number of genes of interest introduced therein and the expression levels.

### BACKGROUND OF THE INVENTION

### Technical Field

The present invention relates to a recombinant cell which enables efficient production of substances of interest such as protein and the like, a method for producing the cell, and a method for producing a substance of interest such as protein and the like by using the cell.

### Background Art

As a recombinant protein production system, variety of methods using prokaryotes or eukaryotes as a host cell are known. According to the recombinant protein production system using a mammalian cell as a host cell, it is possible to subject proteins derived from higher animals including human to post-translational modifications such as sugar chain addition, folding, and phosphorylation in a manner more similar to those produced in vivo.

The post-translational modifications are necessary to reproduce physiological activities, which are inherent to protein, in the recombinant protein. Thus, the protein production system using a mammalian cell as a host cell is used as a production system of recombinant protein used in medicines and the like, which particularly requires such physiological activities.

In order to construct the protein production system, there has been proposed a technique, wherein a gene of interest is integrated specifically into a target gene locus in the cell genome to efficiently obtain a recombinant cell which can express the protein stably for a long period.

An example of the target gene includes a hypoxanthine-guanine phosphoribosyltransferase (hprt) gene. The hprt gene is known as one of the housekeeping genes present on the long arm of X chromosome of humans and the like. Cells after knockout of the hprt gene exhibit resistance to a drug, 6-thioguanine (6TG), and therefore negative selection is performed easily.

It has been reported that, by introducing, via a recombinant vector, a gene of interest into the hprt gene locus of HT1080 cell line derived from human male, recombinant cells were obtained which could express protein stably for a long period in the absence of a selection drug (JP2007-325571A and Koyama Y. et al. (2006) Biotechnology And Bioengineering, 95, 1052-1060).

Further, some of the present inventors has proposed a method of producing a substance of interest such as protein and the like by preparing mammalian cells having multiple copies of an exogenous gene of interest integrated into the hprt gene locus and by culturing the cells (WO 2009/022656). Further, there has been proposed a technique of applying this method to CHO cells (WO2011/040285).

As far as the present inventors know, the above conventional technologies showed phenomena that, when multiple gene expression units were introduced into the same site of chromosome, the gene introduced did not express itself in an expected level or the expression deteriorated with time.

On the other hand, as a technique for amplifying genes, there has been proposed, for example, a method using a recombinase Cre and a target site of Cre, the target site comprising a spacer region and left and right arm regions arranged respectively on the left and right thereof (JP 2009-189318A; Kameyama, Y. et al., Biotechnol. Bioeng. 105, 1106 (2010); and Inao, T., et al., Journal of Bioscience and Bioengineering 120, 99-106, 2015). This method is called an accumulative gene integration system (AGIS) and it is mentioned that, according to this method, amplification of the gene of interest can be performed on a chromosome of an animal cell and that the cells obtained can be used for mass production of recombinant protein.

### CITATION LIST

### Patent Literature

[PTL 1] JP 2007-325571A

[PTL 2] WO 2009/022656

[PTL 3] WO2011/040285

[PTL 4] JP 2009-189318A

### None Patent Literature

[NPL 1] Koyama Y. et al., Biotechnology and Bioengineering, 95, 1052-1060 (2006)

[NPL 2] Kameyama, Y. et al., Biotechnol. Bioeng. 105, 1106 (2010)

[NPL 3] Inao, T., et al., Journal of Bioscience and Bioengineering 120, 99-106 (2015)

### SUMMARY OF THE INVENTION

We have now found that a system, which enables production of a substance of interest such as recombinant protein and the like more stably for a long period, can be realized by, when introducing multiple exogenous genes of interest, controlling chain lengths and sequences among the expression units of the multiple genes of interest. In particular, the present inventors have found that a strong positive linear correlation is shown between the number of the genes of the interest introduced and the expression level. The present invention is based on such findings.

Thus, the object of the present invention is to provide a recombinant cell which enables efficient production of a substance of interest such as protein and the like, a method for producing the cells, and a method for producing the substance of interest by using the cells.

And, a cell according to the present invention is a mammalian cell comprising multiple expression units which comprise an exogenous gene of interest and which are integrated therein, wherein the number of the multiple expression units is 3 or more; and further comprising exogenous gap elements which are provided among the multiple expression units wherein the gap elements have chain lengths of 0.5 kbp or more and 20 kbp or less, and the gap elements are mutually different sequences.

Further, a method for producing the cell according to the present invention is characterized in that the multiple expression units and gap elements are integrated by AGIS.

Furthermore, a method for producing a substance of interest such as protein and the like according to the present invention comprises a step of culturing the cells according to the present invention.

### BRIEF DESCRIPTION OF THE DRAWINGS

FIG. 1 is a schematic diagram of a trastuzumab heavy chain expression vector in Step (1-1), where (1) is a general view of the vector and (2) is a trastuzumab heavy chain expression unit which has been cut out.

FIG. 2 is a schematic diagram of a trastuzumab light chain expression vector in Step (1-2), where (1) is a general view of the vector and (2) is a trastuzumab light chain expression unit which has been cut out.

FIGS. 3A, 3B and 3C are schematic diagrams of a method for preparing a x1 AGIS trastuzumab expression vector (vector 1) in Step (1-3).

FIGS. 4A, 4B and 4C are schematic diagrams of a method for preparing a x2 AGIS trastuzumab expression vector (vector 2) in Step (1-4).

FIGS. 5A, 5B and 5C are schematic diagrams of a method for preparing a x3 AGIS trastuzumab expression vector (vector 3) in Step (1-5).

FIG. 6 is a schematic diagram of a CHO hprt AGIS origin vector in Step (2-1).

FIG. 7A and FIG. 7B illustrate: a schematic diagram of integration of CHO hprt AGIS origin vector into the hprt gene locus of a CHO cell by homologous recombination in Step (2-3); and a result of confirmation of the integration. It is a schematic diagram which illustrates a relationship among a CHO hprt AGIS origin vector, a hprt gene region of a CHO cell, and a DNA which has become an index of genomic PCR of a recombinant CHO cell.

FIG. 7C is a result of electrophoresis for confirmation of vector integrationby PCR using genomic DNA as a template.

FIG. 8A and FIG. 8B illustrate: a schematic diagram of integration of vector 1 into a CHO cell by accumulative integration in Step (2-4), the CHO cell having a CHO hprt AGIS origin vector integrated therein; and a result of confirmation of the integration. It is a schematic diagram which illustrates a relationship among vector 1, a CHO hprt AGIS origin vector region of a CHO cell having a CHO hprt AGIS origin vector integrated therein, and a DNA which has become an index of genomic PCR of a recombinant CHO cell.

FIG. 8C is a result of electrophoresis for confirmation of vector integration by PCR using genomic DNA as a template.

FIG. 9A and FIG. 9B illustrate: a schematic diagram of integration of vector 2 into a CHO cell by accumulative integration, the CHO cell having vector 1 integrated therein; and a result of confirmation of the integration. It is a diagram which illustrates a relationship among vector 2, a vector 1 region of a CHO cell having vector 1 integrated therein, and a DNA which has become an index of genomic PCR of a recombinant CHO cell.

FIG. 9C is a result of electrophoresis for confirmation of vector integration by PCR using genomic DNA as a template.

FIG. 10A and FIG. 10B illustrate: a schematic diagram of integration of vector 3 into a CHO cell by accumulative integration, the CHO cell having vector 1 and vector 2 integrated therein; and a result of confirmation of the integration. It is a diagram which illustrates a relationship among vector 3, vector 1 and vector 2 regions of a CHO cell having vector 1 and vector 2 integrated therein, and a DNA which has become an index of genomic PCR of a recombinant CHO cell.

FIG. 10C is a result of electrophoresis for confirmation of vector integration by PCR using genomic DNA as a template.

FIG. 11A illustrates results of quantification of copy numbers of trastuzumab heavy chain and trastuzumab light chain in a CHO cell having vector 1 integrated therein, a CHO cell having vector 1 and vector 2 integrated therein, and a CHO cell having vector 1, vector 2, and vector 3 integrated therein. It illustrates results of quantification of copy numbers of trastuzumab heavy chain DNA and trastuzumab light chain DNA in the chromosome of each cell having a vector(s) integrated therein.

FIG. 11B illustrates results of quantification of copy numbers of trastuzumab heavy chain and trastuzumab light chain in a CHO cell having vector 1 integrated therein, a CHO cell having vector 1 and vector 2 integrated therein, and a CHO cell having vector 1, vector 2, and vector 3 integrated therein. It illustrates results of quantification of copy numbers of trastuzumab heavy chain mRNA and trastuzumab light chain mRNA in the chromosome of each cell having a vector(s) integrated therein.

FIG. 12A is a confirmation result of long-term stability in the antibody expression levels of a CHO cell having vector 1 integrated therein, a CHO cell having vector 1 and vector 2 integrated therein, and a CHO cell having vector 1, vector 2, and vector 3 integrated therein.

FIG. 12B is a result of addition, to FIG. 12A, of data of antibody expression levels by a CHO cell having vector 1, vector 2, and vector 3 integrated therein after subculture for 112 days, 168 days, and 224 days.

FIGS. 13A, 13B and 13C are schematic diagrams of a method for preparing a x2 AGIS gap element A vector in Comparative Example 2.

FIG. 14A and FIG. 14B illustrate: a schematic diagram of integration of a x2 AGIS gap element A vector into a CHO cell by accumulative integration, the CHO cell having vector 1 integrated therein; and a result of confirmation of the integration. It is a diagram which illustrates a relationship among a x2 AGIS gap element A vector, a vector 1 region of a CHO cell having a vector 1 integrated, and a DNA which has become an index for genomic PCR of a recombinant CHO cell.

FIG. 14C is a result of electrophoresis for confirmation of vector integration by PCR using genomic DNA as a template.

FIG. 15 illustrates a confirmation result of long-term stability in antibody expression level of a CHO cell having vector 1 and a x2 AGIS gap element A vector integrated therein.

FIGS. 16A, 16B and 16C are schematic diagrams of a method for preparing a x2 AGIS gap element B500 vector in Example 3.

FIG. 17A and FIG. 17B illustrate: a schematic diagram of integration of a x2 AGIS gap element B500 vector into a CHO cell by accumulative integration, the CHO cell having vector 1 integrated; and a result of confirmation of the integration. It is a diagram which illustrates a relationship among a x2 AGIS gap element B500 vector, a vector 1 region of a CHO cell having vector 1 integrated, and a DNA which has become an index for genomic PCR of a recombinant CHO cell.

FIG. 17C is a result of electrophoresis for confirmation of vector integration by PCR using genomic DNA as a template.

FIG. 18 is a confirmation result of long-term stability in antibody expression level of a CHO cell having vector 1 and x2 AGIS gap element B500 vector integrated therein.

FIGS. 19A, 19B and 19C are schematic diagrams of a method for preparing a x2 AGIS gap element C500 vector in Example 4.

FIG. 20A and FIG. 20B illustrate: a schematic diagram of integration of a x2 AGIS gap element C500 vector into a CHO cell by accumulative integration, the CHO cell having vector 1 integrated therein; and a result of confirmation of the integration. It is a diagram that illustrates a relationship among a x2 AGIS gap element C500 vector, a vector 1 region of a CHO cell having vector 1 integrated therein, and a DNA which has become an index for genomic PCR of a recombinant CHO cell.

FIG. 20C is a result of electrophoresis for confirmation of vector integration by PCR using genomic DNA as a template.

FIG. 21 is a confirmation result of long-term stability in antibody expression level of a CHO cell having vector 1 and x2 AGIS gap element C500 vector integrated therein.

FIGS. 22A, 22B and 22C are schematic diagrams of a method for preparing a x1 AGIS gap element A500 vector in Example 5.

FIGS. 23A, 23B and 23C are schematic diagrams of a method for preparing a x2 AGIS gap element C500-B500 vector in Example 5.

FIG. 24A and FIG. 24B are a schematic diagram of integration of x1 AGIS gap element A500 vector, in Example 5, into a CHO cell by accumulative integration, the CHO cell having a CHO hprt AGIS origin vector integrated therein. It is a diagram that illustrates a relationship between a x1 AGIS gap element A500 vector and a CHO hprt AGIS origin vector region of a CHO cell having CHO hprt AGIS origin vector integrated therein.

FIG. 25A and FIG. 25B illustrate: a schematic diagram of integration of a x2 AGIS gap element C500-B500 vector into a CHO cell by accumulative integration, the CHO cell having x1 AGIS gap element A500 vector integrated therein; and a result of confirmation of the integration. It is a diagram that illustrates a relationship among a x2 AGIS gap element A vector, a x1 AGIS gap element A500 region of CHO cell having x1 AGIS gap element A500 vector integrated therein, and a DNA which has become an index for genomic PCR of a recombinant CHO cell.

FIG. 25C is a result of electrophoresis for confirmation of vector integration by PCR using genomic DNA as a template.

FIG. 26 is a confirmation result of long-term stability in antibody expression level of a CHO cell having x1 AGIS gap element A500 vector and x2 AGIS gap element C500-B500 vector integrated therein.

### DETAILED DESCRIPTION OF THE INVENTION

Gap Element

The “expression unit” according to the present invention, which is integrated into the mammalian cell, is the structure which is not limited as long as the structure enables expression of an exogenous gene of interest, i.e., a substance of interest such as protein, nucleic acid, or the like. In particular, in the case of protein, it preferably refers to a unit provided with a promoter, a coding region of the protein of interest (from the transcription start point to the stop codon), and a poly A sequence. Details of compositional elements of an expression unit such as a gene of interest and the like will be described later.

The “promoter” is a string of DNA sequence having a promoter function, which is obtained from a genomic DNA or a viral genome. Alternatively, the promoter can be a sequence containing a region which has a promoter function, which is obtained from a known DNA construct by cutting out with a suitable restriction endonuclease or by taking out after amplification by a PCR reaction.

The mammalian cell according to the present invention has 3 or more expression units integrated therein and, further, has “exogenous gap elements” among the multiple expression units. In the present invention, the gap elements have a chain length of 0.5 kbp or more, and the gap elements are mutually different sequences. Due to presence of such gap elements among the expression units, there is stably obtained an improvement in expression in proportion to the number of the expression units introduced therein.

While the expression level of the protein of interest is expected to be proportional to the number of genes introduced, in practice, a decrease in the expression level with time has been observed with conventional techniques, even though an expression level proportional to the number of genes is obtained immediately after the introduction. In the present invention, a stable expression level can be maintained for a long period, for example, for 50 days or more, for 100 days or more under preferable conditions, for 200 days under more preferable conditions, and for 300 days under most preferable conditions. The term “stable expression level” means that production relative to that immediately after establishment of the cell is maintained preferably at 70% or more, more preferably at 80% or more, and most preferably at 90% or more.

According to a preferred embodiment of the present invention, the lower limit of chain length of a gap element is preferably 0.5 kbp, more preferably 1 kbp, further more preferably 1.5 kbp, and most preferably 2 kbp. In addition, the upper limit of length of the gap element is preferably 20 kbp, more preferably 10 kbp, further more preferably 5 kbp, and most preferably 3 kbp. A preferable range of chain length of the gap element is determined by selecting and combining the lower limit value and the upper limit value.

According to one embodiment of the present invention, it is advantageous for stable long-term production of recombinant protein that at least one of the multiple gap elements is designed to have a chain length of 1 kbp or more.

Furthermore, in the present invention, the multiple gap elements are so designed as to be mutually different sequences, but mutual homology is designed to be preferably 99.5% or less, more preferably 95% or less, further more preferably 90% or less, further more preferably 87.5% or less, further more preferably 85% or less, further more preferably 82.5% or less, further more preferably 80% or less, further more preferably 77.5% or less, further more preferably 75% or less, further more preferably 72.5% or less, further more preferably 70% or less, further more preferably 67.5% or less, further more preferably 65% or less, further more preferably 62.5% or less, further more preferably 60% or less, further more preferably 57.5% or less, further more preferably 55% or less, further more preferably 52.5% or less, and most preferably 50% or less.

In the present invention, homology is determined in the following manner. That is, of two gap elements one want to compare, homology of an arbitrary sequence in the longer sequence, which has the same length as the full length of the shorter sequence, is determined successively, and the maximum value of homology obtained is defined as the homology. Specific methods for determining homology include a method of using a homology search function of genetic information software, and the like. More specifically, the gap elements can be compared by using (base sequence)×(base sequence) homology search of GENETYX Ver. 10 software (GENETYX CORPORATION).

In the present invention, the gap element is exogenous. The term “exogenous” is used to mean not only a sequence not intrinsically possessed by the mammalian cell into which the gap element is integrated but also a sequence which, even though it is a sequence possessed by the mammalian cell into which the gap element is integrated, should not intrinsically be present at the position.

In the present invention, the gap element does not possess a particular function. In other words, the gap element can be defined as a sequence between two expression units. However, the gap element may contain a sequence having a specific function, as long as it does not intrinsically exert a bad influence on expression of a substance of interest such as protein and the like.

Therefore, according to one embodiment of the present invention, the gap element can be composed of, for example, a sequence arbitrarily obtained from chromosomes of other cells and organisms, a sequence prepared by synthesis based on a sequence designed on a computer, a sequence which constitutes a vector other than the expression unit (such as a simple vector skeleton, a selection marker, a reporter such as GFP, and the like), vector parts remaining as a result of the AGIS (accumulative gene integration system) which will be described later, a marker sequence, and the like.

Sequence as Site of Integration Into Animal Cell

In the present invention, the site of integration of the expression unit can be appropriately determined by considering techniques of integrating the expression unit and the like. For example, there can be mentioned a method of integrating the expression unit into a stable gene locus, a method of integrating the expression unit randomly into a suitable site, a method of integrating the expression unit into an extra chromosomal vector, and the like. According to a preferred embodiment of the present invention, the integration technique includes the method of integrating the expression unit into a stable gene locus. Here, the stable gene locus includes, for example, the hypoxanthine-guanine phosphoribosyltransferase (hprt) gene locus, the ROSA 26 gene locus region, the PhiC31 pseudo attP site, and the like. Further, in the case of random insertion, there is mentioned a method of screening the cell having an expression unit integrated incidentally at a stable site on the chromosome. Furthermore, the integration site may not be on the chromosome, but may be, for example, on an artificial chromosome such as human artificial chromosome (HAC vector), mouse artificial chromosome (MAC vector), and the like.

According to a preferred embodiment of the present invention, there is used the hprt gene locus as an integration site. Integration of an expression unit into this target region inactivates functions of the hprt gene by inhibiting transcription and expression of the hprt gene, and is preferable for obtaining a recombinant cell efficiently by negative selection using 6-TG and the like.

Furthermore, in the present invention, the target region can be appropriately determined in the hprt gene locus, as long as it does not interfere with expression of the gene of interest. A specific sequence can be determined with reference to descriptions in WO 2011/040285, which is incorporated by reference in its entirely.

Mammalian Cell

Further, in the present invention, the mammalian cells used as the host cells are preferably one derived from a Chinese hamster, and there can be used a cell line established from cells taken out from an organ thereof such as, for example, lung, ovary, kidney, and the like. Specific examples of such Chinese hamster cells include, for example, CHO cells, CHO-K1, CHO-K1SV (Lonza), CHO-S (Life Technologies), DG44 (Life Technologies), DXB11, CHOZN GS (Merck), CHOZN DHFR (Merck), derivative cell lines of these, and the like.

In addition, as the mammalian cells, also usable is one derived from human, mouse, monkey, and the like. For example, specific examples of cells derived from human include HEK 293, PER.C6 (Crucell), HT 1080, and the like. Further, specific examples of cells derived from the mouse include NS0, BHK, SP2/0, SP2/0-AG14, and the like. As an example of the cells derived from monkey, there can be used COS and the like.

Gene of Interest and Expression Unit

Furthermore, in the present invention, the gene of interest preferably encodes protein useful as a medicine. The gene, whether it is a sequence derived from cDNA or a structural gene containing a natural intron derived from genomic DNA, can be suitably used. The protein, which the gene encodes, specifically includes antibodies, enzymes, cytokines, hormones, coagulation factors, regulatory proteins, receptors, functional factors, and the like. However, preferable are monoclonal antibodies, polyclonal antibodies, erythropoietins, tissue plasminogen activators (t-PA), granulocyte-colony stimulating factors (G-CSF), and the like.

According to one embodiment of the present invention, a sequence length of the gene which encodes protein is preferably 200 to 8000 bp, more preferably 300 to 3000 bp, and most preferably 400 to 2500 bp.

As for the kind of protein, the protein produced by mammalian cells is sometimes intended to be generated after being subjected to post-translational modifications characteristic of the mammalian cells. In such an embodiment, the protein is preferably a glycoprotein. The glycoprotein specifically includes: monoclonal antibodies such as trastuzumab, rituximab, panitumumab, pertuzumab, obinutuzumab, bevacizumab, tocilizumab, adalimumab, ipilimumab, daratumumab, nivolumab, atezolizumab, pembrolizumab, mogamulizumab, brentuximab, ixekizumab, secukinumab, brodalumab, omalizumab, evolocumab, denosumab, idarucizumab, alirocumab, dinutuximab, reslizumab, alemtuzumab, briakinumab, ocrelizumab, romosozumab, bezlotoxumab, sarilumab, burosumab, benralizumab, sapelizumab, lebrikizumab, farletuzumab, gantenerumab, patritumab, inebilizumab, adecatumumab, tildrakizumab, fasinumab, margetuximab, bococizumab, dupilumab, erenumab, bimagrumab, durvalumab, galcanezumab, risankizumab, andecaliximab, avelumab, vadastuximab, begelomab, suptavumab, depatuxizumab, opicinumab, tanezumab, ocaratuzumab, coltuximab, pamrevlumab, lirilumab, fulranumab, teprotumumab, urelumab, bleselumab, landogrozumab, neihulizumab, foralumab, anrukinzumab, codrituzumab, icrucumab, glembatumumab, ganitumab, amatuximab, ensituximab, namilumab, ponezumab, dectrekumab, isatuximab, claudiximab, olokizumab, mavrilimumab, ficlatuzumab, monalizumab, crenezumab, diridavumab, demcizumab, lenzilumab, seribantumab, blosozumab, ontuxizumab, lodelcizumab, clazakizumab, polatuzumab, simtuzumab, denintuzumab, anetumab, nemolizumab, indatuximab, imalumab, otlertuzumab, tarextumab, lulizumab, tesidolumab, emibetuzumab, abrilumab, brazikumab, emactuzumab, lumretuzumab, mirvetuximab, varlilumab, cabiralizumab, trevogrumab, evinacumab, pidilizumab, rovalpituzumab, rinucumab, sacituzumab, theralizumab, bimekizumab, ublituximab, leucotuximab, vesencumab, ulocuplumab, vandortuzumab, sonepcizumab, atinumab, flanvotumab, brontictuzumab, vantictumab, concizumab, sevacizumab, enoblituzumab, ralpancizumab, necitumumab, ofatumumab, ramucirumab, elotuzumab, natalizumab, vedolizumab, belimumab, eculizumab, palivizumab, epratuzumab, tremelimumab, tralokinumab, etrolizumab, sirukumab, olaratumab, risankizumab, ligelizumab, panobacumab, tisotumab, cetuximab, golimumab, infliximab, siltuximab, ustekinumab, mepolizumab, canakinumab, basiliximab, daclizumab, solanezumab, anifrolumab, gevokizumab, guselkumab, elgemtumab, enfortumab, crotedumab, oleclumab, gerilimzumab, nesvacumab, parsatuzumab, perakizumab, narnatumab, enavatuzumab, sofituzumab, enoticumab, pateclizumab, tovetumab, pinatuzumab, fezakinumab, placulumab, samalizumab, ozanezumab, vatelizumab, ascrinvacumab, dacetuzumab, carlumab, fresolimumab, imgatuzumab, onartuzumab, oxelumab, and the like; antibody fusion proteins such asetanercept, abatacept, belatacept, aflibercept, rilonacept, efraloctocog, eftrenonacog, dulaglutide, atacicept, luspatercept, baminercept, asinercept, dalantercept, sotatercept, ipafricept, ramatercept, asphotase, and the like; recombinant glycoproteins such as erythropoietin, darbepoetin, alteplase, anistreplase, monteplase, tenecteplase, reteplase, octacog, rurioctocog, moroctocog, turoctocog, susoctocog, simoctocog, nonacog, trenonacog, eptacog, lenograstim, imiglucerase, dolnase, agalsidase, laronidase, alglucosidase, velaglucerase, galsulphase, idursulfase, taliglucerase, hyaronidase, elosulfase, follitropin, thyrotropin, cerliponase, and the like; bispecific antibodies such as emicizumab, vanucizumab, istiratumab, pasotuximab, duligotuzumab, duvortuxizumab, and the like; vaccines containing sugar chains and virus like particles (VLP); or the like.

Further, proteins having no sugar chain include: ScFv antibody, Fab antibody, and modified or fused proteins thereof; functional factors such as ATF4, ATF6, VAMP8, Snap 23, and the like; reporters such as GFP, DsRed, CFP, YFP, SEAP, and the like; and receptors such as EGFR, VEGF, PDGF and the like, vaccines containing no sugar chain, virus-like particles, and the like.

Furthermore, in the present invention, the gene of interest preferably encodes: functional nucleotides such as antisense, siRNA, aptamer, miRNA antisense, miRNA mimic, decoy, CpG oligo, and the like; and functional nucleotides to be fused with proteins to constitute nucleotide fusion proteins.

Also, in the present invention, the expression unit is preferably integrated as an expression unit containing elements required for the expression such as a promoter sequence, a transcription terminator, and the like. Therefore, according to one embodiment of the present invention, the expression unit contains at least a promoter sequence and a transcription terminator.

In addition, the promoter and the transcription terminator can be determined appropriately depending on the kind, nature, and the like of the gene of interest, and suitable examples of such a promoter sequence include, for example, CHEF1 promoter, hEF1 promoter, CMV promoter, SV40 promoter, CAG promoter, CAGG promoter, ribosomal protein promoter, heat shock protein promoter, U6 promoter, tetracycline-inducible promoter, chloramphenicol-inducible promoter, and the like. Also, suitable examples of the transcription terminator include, for example, BGH poly-A signal sequence, SV40 poly-A signal sequence, and the like.

Further, as elements required for the expression other than the promoter sequence and the transcription terminator, there can be used one by appropriately selecting from, for example, regulatory elements for efficiently expressing the gene of interest, which include, for example, enhancer, IRES (internal ribosome entry site) sequence, UCOE, STAR, MAR, Power express element, and the like. The regulatory element can be arranged depending on its nature at a suitable site in the expression unit. These elements required for the expression are preferably selected in consideration of productivity and the like of the substance of interest such as protein and the like.

According to one embodiment of the present invention, different genes of interest can be integrated in one expression site. It is a remarkable advantage of the present invention that such expression of multiple kinds of genes of interest can be produced stably for a long period.

Integration of Multiple Expression Units and Gap Elements

In the present invention, the method for introducing the expression unit and gap element is not limited as long as the method can integrate a plurality of them. However, there can be mentioned a method of introducing a plurality of them in stages and a method of introducing a plurality of them simultaneously.

The method for introducing the expression units and gap elements in stages include, for example, a method of using a recombinase such as Cre, φC31, R4, Flp, and the like, and a method of using a genome editing technology represented by TALEN, CRISPR/Cas9, and the like. According to a preferred embodiment of the method of using Cre recombinase, the method is preferably performed by accumulative gene integration system (AGIS) as disclosed by Japanese Patent Literature 2009-189318, which is incorporated by reference in its entirely.

The method for introducing multiple expression units and gap elements simultaneously includes, for example, a method where full length of these are chemically synthesized and introduced.

AGIS (Accumulative Gene Integration System)

This method enables repeated integration of a gene of interest into a chromosome by using recombinase Cre and variant LoxP sequences. In other words, this is a method which enables amplification of the gene of interest on the chromosome. This method uses the fact that, by combining characteristics of both arm mutation and spacer mutation of LoxP, an independently irreversible insertion reaction occurs Cre-dependently, and the method is so designed that the target site which can be integrated returns to an initial state after two integration reactions. Herewith, it becomes possible, in principle, to repeat the gene integration reaction infinitely. Specifically, the method is:

a gene recombination method for performing two or more recombinations on a chromosome, comprising use of recombinase Cre and a target site of Cre comprising a spacer region, and a left arm region and a right arm region arranged on the left and right thereof, respectively, the method comprising the steps of providing:

(A) target site 1 comprising spacer region 1, one arm region having a mutation, and the other arm region which is a wild type, the target site being integrated into a host chromosome;

(B) an integration cassette comprising gene 1, and target site 2 and target site 3 which are linked respectively to both sides of gene 1, wherein the target site 2 comprises the spacer region 1, one arm region having a mutation on a side opposite to the target site 1 and close to the gene 1, and the other arm region which is a wild type, and the target site 3 comprises spacer region 2 which can function independently of the spacer region 1, one arm region having a mutation on the same side as the target site 1 and close to the gene 1, and the other arm region which is a wild type; and

(C) a substitution cassette comprising gene 2, and target site 4 and target site 5 which are linked respectively to both sides of the gene 2, wherein the target site 4 comprises the spacer region 2, one arm region having a mutation on a side opposite to the target site 1 and close to the gene 2, and the other arm region which is a wild type, and the target site 5 comprises spacer region 3 (where the spacer region 3 can function in the same manner as the spacer region 1 or independently of each of the spacer regions 1 and 2), one arm region having a mutation on the same side as the target site 1 and close to the gene 2, and the other arm region which is a wild type;

and steps of:

(1) reacting the target site 1 and the integration cassette by action of Cre to obtain a reaction product; and

(2) reacting the reaction product and the substitution cassette by action of Cre.

In the above, preparation of the “(A) target site 1 comprising spacer region 1, one arm region having a mutation, and the other arm region on other side which is a wild type, the target site being integrated into a host chromosome,” that is, integration of an integration origin into the chromosome can be performed by: (1) a method of introducing the integration origin at a specific site of an animal cell by homologous recombination using, for example, a homology arm vector; (2) a method of introducing the integration origin at a specific site by a gene editing technology; (3) a method of screening the cell having the integration origin introduced incidentally at a suitable site by random insertion.

Vector

In the present invention, the multiple expression units and gap elements are integrated into the vector and thereby can be integrated into a mammalian cell genome. Such a vector system includes plasmid vector, cosmid vector, phage vector, artificial chromosome vector, or the like.

The vector can be constructed suitably by combining a restriction endonuclease cleavage reaction and a ligation reaction. For example, the vector can be constructed by: having restriction endonuclease recognition sequences contained at both ends of each constituent unit; performing a cleavage reaction with a restriction endonuclease for the recognition sequence; removing unnecessary DNA sequences (sequence for engineering in E. coli, and the like) by a treatment such as gel excision and the like; and performing a ligation reaction of the units obtained.

A vector DNA constructed by the ligation reaction is purified by extraction with phenol-chloroform and the like, and can be propagated in a host cell such as E. coli, yeast, and the like selected in consideration of the kind of the vector and the like.

Transfection of Vector

As a method for introducing the vector, there can suitably be utilized generally used methods. For example, there can be mentioned calcium phosphate transfection, electroporation, microinjection, a DEAE-dextran method, a method of using a liposome reagent, lipofection using a cationic lipid, a method of using a virus vector, and the like. Here, when the vector is of a cyclic form, it can be linearized by a publicly-known method before being introduced into a cell.

Furthermore, depending on the nature of the vector and the substance of interest such as protein and the like, and in consideration of acquisition efficiency of the recombinant cells and the like, there can also be appropriately applied: a site-specific integration system utilizing a recombinase such as Cre/LoxP system, Flp/FRT system, and the like; or a method of using genome editing technology represented by virus-derived integrase, virus vector, TALEN, CRISPR/Cas9, and the like. Such embodiments are also included in the present invention.

Selection of Cell Line

Further, in the production method according to the present invention, it is preferable to perform cell selection after the transfection. The selection step enables selection of cells with high accuracy by utilizing a method of using a marker gene, a method of selecting the cells with the expression level of the gene of interest or gene product as an index, and the like.

Also, in the production method according to present invention, after obtaining the recombinant cells, they can be cultured even under a serum-free condition by acclimatizing them to a Chemical Defined medium and the like. Such an acclimatization condition can be appropriately determined depending on the state of the recombinant cells.

Production Method of Substance of Interest

Further, according to other embodiment of the present invention, there is provided a method for producing a substance of interest such as protein and the like, comprising preparing the recombinant cells and culturing the cells to produce the substance of interest such as protein and the like. According to the method above, the substance of interest such as protein and the like can be obtained efficiently and stably.

As a medium in the culturing step, there can be appropriately selected a publicly known medium depending on the state of the recombinant cells, but the medium is preferably a serum-free medium. Further, the medium is preferably one having no selection drug added in consideration of culturing cost and purification cost. A preferable example of the medium includes a Chemical Defined medium and the like.

As the culturing method, there can be applied a publicly known method such as a batch culture method, a fed-batch culture method, a perfusion culture method, and the like.

### EXAMPLES

The present invention will be described in detail by reference to the following examples, but the present invention is not limited to these examples.

In the following examples, LoxP sequences used were as follows.

| Name | Base Sequence (5′→3′) |
|---|---|
| LoxPwt | ATAACTTCGTATA GCATACAT TATACGAAGTTAT |
| LoxPsp8 | ATAACTTCGTATA aagtatcc TATACGAAcggta |
| LoxPsp8′ | taccgTTCGTATA aagtatcc TATACGAAGTTAT |
| LoxPsp10 | ATAACTTCGTATA accataat TATACGAAcggta |
| LoxPsp10′ | taccgTTCGTATA accataat TATACGAAGTTAT |

### Preparation and Evaluation of Cells in Examples 1 and 2 and Comparative Example 1

### Step 1: Preparation of Accumulatively Integration Vector Containing Gap Elements of Mutually Different Sequences Among Expression Units

### Step (1-1) Preparation of Trastuzumab Heavy Chain Expression Vector

To a secretion signal peptide (METDTLLLWVLLLWVPGSTGD) (SEQ ID NO: 1) of mouse Igκ was bound the amino acid sequence of trastuzumab heavy chain (obtained from trastuzumab (gene recombinant) examination report published on Jan. 16, 2008 by Pharmaceuticals and Medical Devices Agency), and a DNA sequence which encodes this sequence was designed. Next, on the 5′ side of the sequence were attached the recognition sequence (underlined part) for restriction endonuclease EcoRI (GAATTCgccgccacc) and a Kozak sequence (lowercase letters), and to the 3′ terminal were attached a stop codon and the recognition sequence (tgaCTCGAG) for restriction endonuclease Xhol. DNA was synthesized based on this sequence to prepare a trastuzumab heavy chain gene fragment.

Subsequently, using pmaxGFP plasmid (amaxa) used as a template, SV40 poly A was amplified by a PCR reaction with a SV40pA-s/SV40pA-a primer set to obtain an SV40 poly A fragment. To the 5′ terminal of the sense primer was attached the recognition sequence (underlined part) for restriction endonuclease Xhol, and to the 5′ terminal of the antisense primer, was attached the recognition sequence (underlined part) for restriction endonuclease Clal.

SV40pA-sprimer:
(SEQ ID NO: 2)
5′-CTCGAGctcgatgagtttggacaaac-3′
SV40pA-aprimer:
(SEQ ID NO: 3)
5′-ATCGATGTCGACAAGCTTTCTAGAACGCGTtaccacatttgta
gaggtt-3′

Next, the genomic DNA of CHO-K1 (ATCC CCL-61) was extracted with MAG Extractor (TOYOBO) and, using this as a template, a PCR reaction with a CHEF1α promoter-s/CHEF1α promoter-a primer set was performed to amplify a CHO endogenous EF1α promoter and to obtain a CHEF1α promoter fragment. To the 5′ terminal of the sense primer was attached the recognition sequence (underlined part) for restriction endonuclease Clal, and to the 5′ terminal of the antisense primer was attached the recognition sequence(underlined part)for restriction endonuclease EcoRI.

CHEF1α promoter-sprimer:
(SEQ ID NO: 4)
5′-ATCGATGTCGACAAGCTTTCTAGAACGCGTccacacaatcaga
accaca-3′
CHEF1α promoter-aprimer:
(SEQ ID NO: 5)
5′-GAATTCgcggtggttttcacaacacc-3′

Then, using pCR-Bluntll-TOPO vector (Life Technologies) as a template, a kanamycin resistance gene, a zeocin resistance gene, and a replication origin were amplified by a PCR reaction with a pUC-s/pUC-a primer set to obtain the plasmid backbone. To each of the 5′-terminal of the sense primer and the 5′-terminal of the antisense primer was attached the recognition sequence (underlined part) for restriction endonuclease Clal.

pUC-s primer:
(SEQ ID NO: 6)
5′-ATCGATGCTTACATGGCGATAGCTAG-3′
pUC-a primer:
(SEQ ID NO: 7)
5′-ATCGATGAATCAGGGGATAACGCAGG-3′

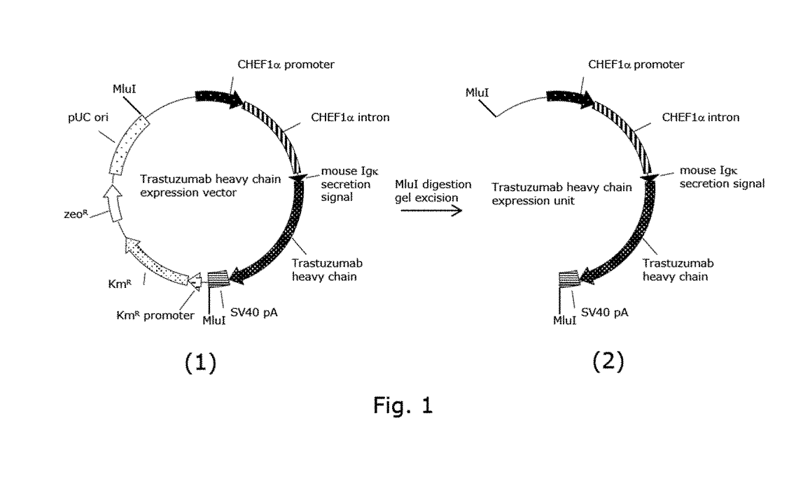

The prepared and obtained CHEF1α promoter fragment, trastuzumab heavy chain gene fragment, SV40 poly A fragment, and plasmid backbone were linked by restriction endonuclease digestion and ligation of the terminals to prepare the trastuzumab heavy chain expression vector of FIG. 1 (1).

The trastuzumab heavy chain expression vector obtained was digested with restriction endonuclease Mlul and subjected to agarose gel electrophoresis, and the trastuzumab heavy chain expression unit of FIG. 1 (2) was extracted.

### Step (1-2) Preparation of Trastuzumab Light Chain Expression Vector

In the same manner as in Step (1-1), to the secretion signal peptide of mouse Igκ was bound the amino acid sequence of trastuzumab light chain (obtained from the same examination report as in Step (1-1)), and a DNA sequence which encodes this sequence was designed. Next, on the 5′ side of the sequence were attached the recognition sequence (underlined part) (GAATTCgccgccacc) for restriction endonuclease EcoRI and a Kozak sequence (lowercase letters), and to the 3′ terminal were attached a stop codon (lowercase letters) and the recognition sequence (underlined part) (tgaCTCGAG) for restriction endonuclease Xhol. DNA was synthesized based on this sequence to prepare a trastuzumab light chain gene fragment.

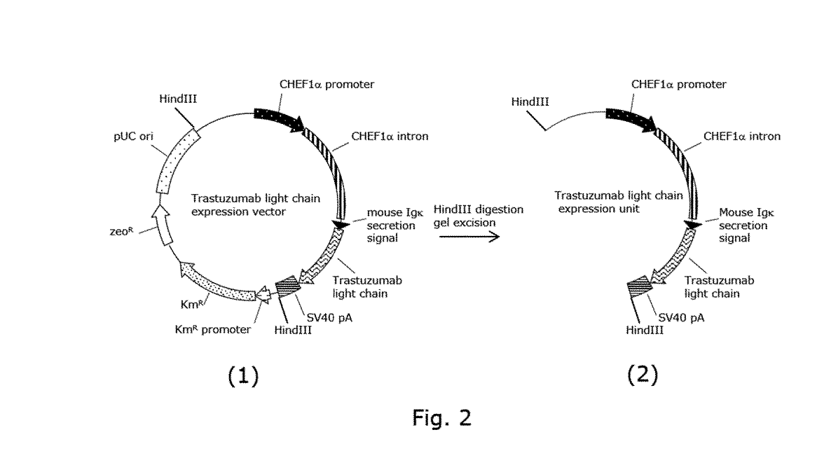

The CHEF1α promoter fragment, SV40 poly A fragment, and plasmid backbone, which were obtained and prepared in Step (1-1), and the trastuzumab light chain gene fragment were linked by restriction endonuclease digestion and ligation of the terminals to prepare the trastuzumab light chain expression vector of FIG. 2 (1).

The thus obtained trastuzumab light chain expression vector was digested with restriction endonuclease Mlul and subjected to agarose gel electrophoresis, and the trastuzumab light chain expression unit of FIG. 2 (2) was extracted.

### Step (1-3) Preparation of x1 AGIS Trastuzumab Expression Vector (Vector 1)

Using pQBl25 plasmid vector (Wako Pure Chemical Industries, Ltd.) as a template, a PCR reaction was performed with a pUC-amp-s1/pUC-amp-a1 primer set to obtain a fragment (1) containing a replication origin and ampicillin resistance gene. To the 5′ terminal of the sense primer were attached the recognition sequence (underlined part) for restriction endonuclease Spel and LoxPsp8′ sequence (lowercase letters) of Table 1, and to the 5′ terminal of the antisense primer was attached the recognition sequence (underlined part) for restriction endonuclease BamHI.

pUC-amp-s1primer:
(SEQ ID NO: 8)
5′-ACTAGTCtaccgttcgtataaagtatcctatacgaagttatTA
GCTTCAGTGAGCAAAAG-3′
pUC-amp-a1primer:
(SEQ ID NO: 9)
5′-GGATCCtcgcgtaggtggcacttttc-3′

Subsequently, using pBApo-EF1α Pur vector (TaKaRa) as a template, a PCR reaction was performed with a puro-s/puro-a primer set to obtain a fragment containing puromycin resistance gene and SV40 poly A. To the 5′ terminal of the sense primer were attached the recognition sequence (underlined part) for restriction endonuclease BamHI and LoxPwt sequence (lowercase letters) of Table 1, and to the 5′ terminal of the antisense primer were attached the recognition sequence (underlined part) for restriction endonuclease Spel and a complementary sequence (lowercase letters) to the LoxPsp10 sequence of Table 1.

puro-s primer:
(SEQ ID NO: 10)
5′-GGATCCataacttcgtatagcatacattatacgaagttatCCA
CCGAGTCAAGCCCACGGT-3′
puro-a primer:
(SEQ ID NO: 11)
5′-ACTAGTAAGCTTACGCGTtaccgttcgtataattatggttata
cgaagttatACGATCATAATCAGCCATAC-3′

The fragment (1) containing a replication origin and ampicillin resistance gene and the fragment containing puromycin resistance gene and SV40 poly A, which were obtained above, were ligated to prepare x1 basic vector.

Next, using genomic DNA of human embryonic kidney cell-derived HEK293 (JCRB ID: 9068) as a template,a dummy sequence of 3130 bp was amplified with a dummy sequence-s/dummy sequence-a primer set. To the 5′ terminal of the sense primer was attached the recognition sequence (underlined part) for restriction endonuclease HindIII, and to the 5′ terminal of the antisense primer was attached the recognition sequence (underlined part) for restriction endonuclease Spel.

Dummy sequence-s primer:
(SEQ ID NO: 12)
5′-AAGCTTGGCAACATAGCGAGACTTCG-3′
Dummy sequence-a primer:
(SEQ ID NO: 13)
5′-ACTAGTGTGTACAGAAGGCTGAAAGG-3′

Furthermore, using genomic DNA of HEK293 as a template, gap element A (SEQ ID NO: 14) of 2051 bp was PCR amplified with a gap element A-s/gap element A-a primer set, and the length of amplification product was confirmed by agarose gel electrophoresis. To the 5′ terminal of the sense primer was added the recognition sequence (underlined part) for restriction endonuclease Mlul, and to the 5′ terminal of the antisense primer was added the recognition sequence (underlined part) for restriction endonuclease HindIII.

Gap element A-s primer:
(SEQ ID NO: 15)
5′-ACGCGTTGAAAGTGTGAGTTCCGGAG-3′
Gap element A-a primer:
(SEQ ID NO: 16)
5′-AAGCTTAACTGCAGCCTATTGGTAGC-3′

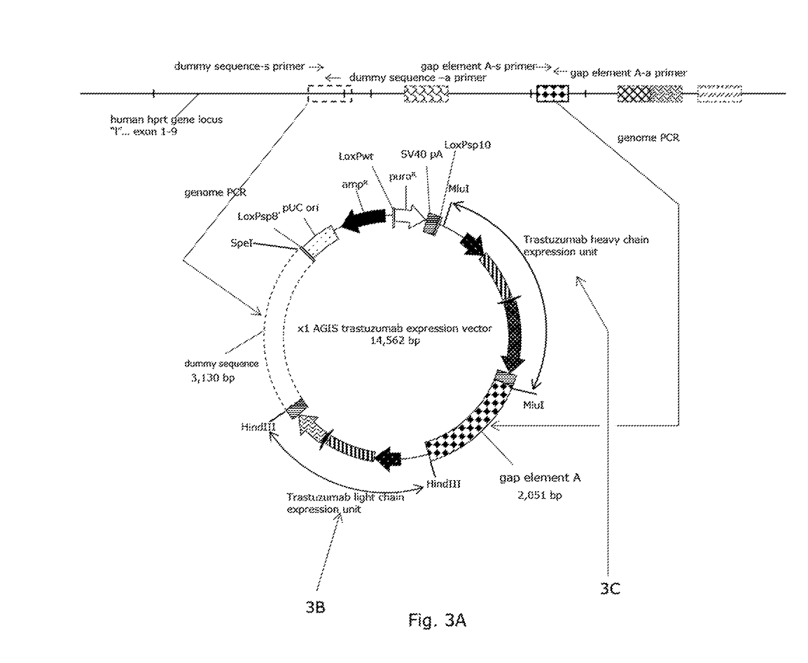

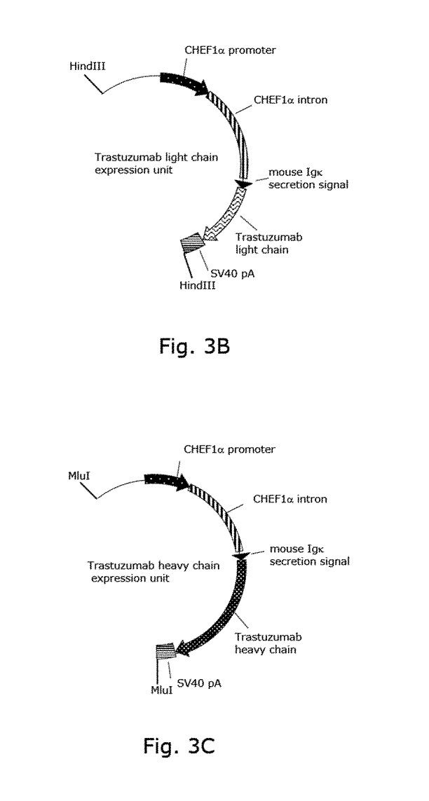

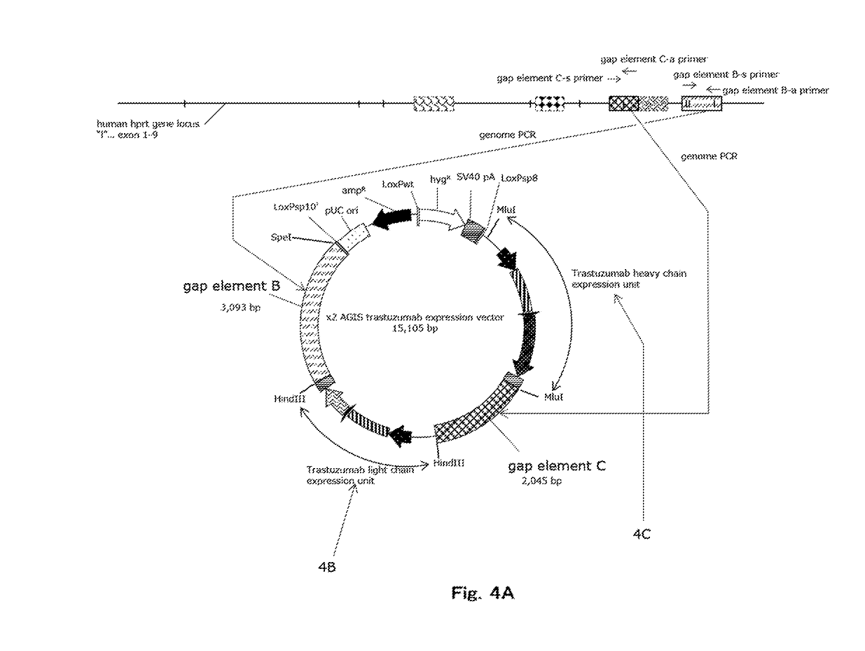

The gap element A was inserted between the Mlul and HindIII sites of the x1 basic vector obtained above. Then, the dummy sequence was inserted between the HindIII and Spel sites, and subsequently the fragment of the trastuzumab heavy chain expression unit of Step (1-1) was inserted in the Mlul site. Finally, the fragment of the trastuzumab light chain expression unit of Step (1-2) was inserted in the HindIII site to prepare the vector 1 shown in FIG. 3A.

### Step (1-4) Preparation of x2 AGIS Trastuzumab Expression Vector (Vector 2)

Using pQBl25 plasmid vector as a template, a PCR reaction was performed with a pUC-amp-s2/pUC-amp-a2 primer set to obtain a fragment (2) containing a replication origin and an ampicillin resistance gene. To the 5′ terminal of the sense primer were attached the recognition sequence (underlined part) for restriction endonuclease Spel and LoxPsp10′ sequence (lowercase letters) of Table 1, and to the 5′ terminal of the antisense primer was attached the recognition sequence (underlined part) for restriction endonuclease BamHI.

pUC-amp-s2 primer:
(SEQ ID NO: 17)
5′-ACTAGTCtaccgttcgtataaccataattatacgaagttatTA
GCTTCATGTGAGCAAAAG-3′
pUC-amp-a2 primer:
(SEQ ID NO: 18)
5′-GGATCCtcgcgTaggtggcacttttc-3′

Subsequently, using pcDNA3.1/Hygro(+) vector (Life Technologies) as a template, a PCR reaction was performed with a Hygro-s/Hygro-a primer set to obtain a fragment containing a hygromycin resistance gene and SV40 poly A. To the 5′ terminal of the sense primer were attached the recognition sequence (underlined part) for restriction endonuclease BamHI and LoxPwt sequence (lowercase letters) of Table 1, and to the 5′ terminal of the antisense primer were attached the recognition sequence (underlined part) for restriction endonuclease Spel and a complementary sequence (lowercase letters) to LoxPsp8 sequence of Table 1.

Hygro-s primer:
(SEQ ID NO: 19)
5′-GGATCCataacttcgtatagcatacattatacgaagttatCCA
AAAAGCTGAACTCACCGC-3′
Hygro-a primer:
(SEQ ID NO: 20)
5′-ACTAGTAAGCTTACGCGTTaccgttcgtataggatactttata
cgaagttatTAGCTCACTCATTAGGCACC-3′

The fragment (2) containing a replication origin and ampicillin resistance gene and the fragment containing hygromycin resistance gene and SV40 poly A, which were obtained above, were ligated to prepare a x2 basic vector.

Next, using genomic DNA of HEK293 as a template, gap element C of 2045 bp was PCR amplified with a gap element C-s/gap element C-a primer set, and the length of amplification product was confirmed by agarose gel electrophoresis. To the 5′ terminal of the sense primer was attached the recognition sequence (underlined part) for restriction endonuclease Mlul, and to the 5′ terminal of the antisense primer was attached the recognition sequence (underlined part) for restriction endonuclease HindIII.

gap element C-s primer:
(SEQ ID NO: 21)
5′-ACGCGTCGTTTTCTCACTGGCTGCTG-3′
gap element C-a primer:
(SEQ ID NO: 22)
5′-AAGCTTACTCTCATAGTACCTGTCCC-3′

Further, using genomic DNA of HEK293 as a template, gap element B (SEQ ID NO: 23) of 3093 bp was PCR amplified with a gap element B-s/gap element B-a primer set, and the length of amplification product was confirmed by agarose gel electrophoresis. To the 5′ terminal of the sense primer was attached the recognition sequence (underlined part) for restriction endonuclease HindIII, and to the 5′ terminal of the antisense primer was attached the recognition sequence (underlined part) for restriction endonuclease Spel.

gap element B-s primer:
(SEQ ID NO: 24)
5′-AAGCTTGCTCATTGACACTCTGTACC-3′
gap element B-a primer:
(SEQ ID NO: 25)
5′-ACTAGTCTCACGTGGAGCAGATAAGC-3′

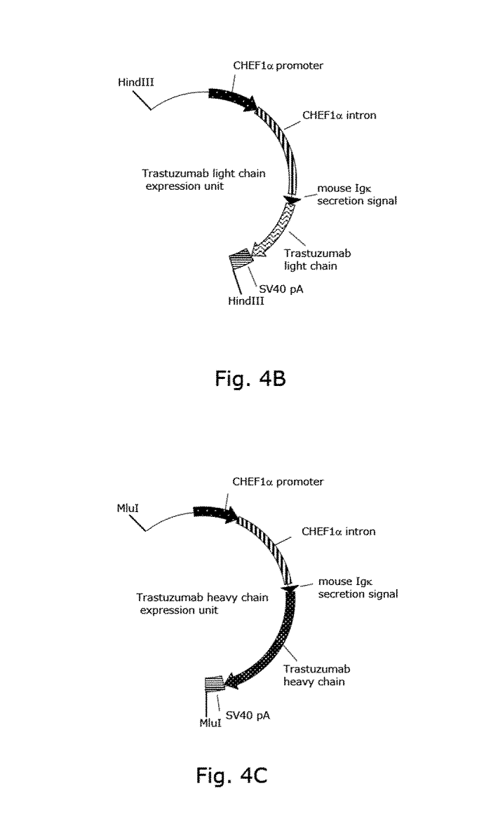

Gap element C (SEQ ID NO: 26) was inserted between the Mlul and HindIII sites of the x2 basic vector obtained above. Then, the gap element B was inserted between the HindIII and Spel sites, and subsequently the fragment of the trastuzumab heavy chain expression unit of Step (1-1) was inserted in the Mlul site. Finally, the fragment of the trastuzumab light chain expression unit of Step (1-2) was inserted in the HindIII site to prepare the vector 2 shown in FIG. 4A.

### Step (1-5) Preparation of x3 AGIS Trastuzumab Expression Vector (Vector 3)

Using pQBl25 plasmid vector as a template, a PCR reaction was performed with a pUC-amp-s3/pUC-amp-a3 primer set to obtain a fragment (3) containing a replication origin and ampicillin resistance gene. To the 5′ terminal of the sense primer were attached the recognition sequence (underlined part) for restriction endonuclease Spel and LoxPsp8′ sequence (lowercase letters) of Table 1, and to the 5′ terminal of the antisense primer was attached the recognition sequence (underlined part) for restriction endonuclease BamHI.

pUC-amp-s3 primer:
(SEQ ID NO: 27)
5′-ACTAGTCtaccgttcgtataaagtatcctatacgaagttatTA
GCTTCATGTGAGCAAAAG-3′
pUC-amp-a3 primer:
(SEQ ID NO: 28)
5′-GGATCCtcgcgTaggtggcacttttc-3′

Subsequently, using pcDNA6/V5-His/lacZ vector (Life Technologies) as a template, a PCR reaction was performed with aBsd-s/Bsd-a primer set to obtain a fragment containing blasticidin resistance gene and SV40 poly A. To the 5′ terminal of the sense primer were attached the recognition sequence (underlined part) for restriction endonuclease BamHI and LoxPwt sequence (lowercase letters) of Table 1, and to the 5′ terminal of the antisense primer were attached there cognition sequence (underlined part) for restriction endonuclease Spel and a complementary sequence (lowercase letters) to the LoxPsp10 sequence of Table 1.

Bsd-s primer:
(SEQ ID NO: 29)
5′-GGATCCataacttcgtatagcatacattatacgaagttatCCG
CCAAGCCCTGTCCCAGGA-3′
Bsd-a primer:
(SEQ ID NO: 30)
5′-ACTAGTAAGCTTACGCGTtaccgttcgtataattatggttata
cgaagttatACGATCATAATCAGCCATAC-3′

The fragment (3) containing a replication origin and ampicillin resistance gene and the fragment containing blasticidin resistance gene and SV40 poly A, which were obtained above, were linked to prepare x3 basic vector.

Next, using genomic DNA of HEK293 as a template, gap element E of 2033 bp was PCR amplified with a gap element E-s/gap element E-a primer set, and the length of amplification product was confirmed by agarose gel electrophoresis. To the 5′ terminal of the sense primer was attached the recognition sequence (underlined part) for restriction endonuclease Mlul, and to the 5′ terminal of the antisense primer was attached the recognition sequence (underlined part) for restriction endonuclease HindIII.

gap element E-s primer:
(SEQ ID NO: 31)
5′-ACGCGTCAACCTACTGTGGCATAGAC-3′
gap element E-a primer:
(SEQ ID NO: 32)
5′-AAGCTTACAGGCAGAGGTTATCCTGG-3′

Further, using genomic DNA of HEK293 as a template, gap element D (SEQ ID NO: 33) of 3001 bp was PCR amplified with a gap element D-s/gap element D-a primer set, and the length of amplification product was confirmed by agarose gel electrophoresis. To the 5′ terminal of the sense primer was attached the recognition sequence (underlined part) for restriction endonuclease HindIII, and to the 5′ terminal of the antisense primer was attached the recognition sequence (underlined part) for restriction endonuclease Spel.

gap element D-s primer:
(SEQ ID NO: 34)
5′-AAGCTTAGATCACAGCTCACTGTGGC-3′
gap element D-a primer:
(SEQ ID NO: 35)
5′-ACTAGTCGGCTGACACCTGTAATCTC-3′

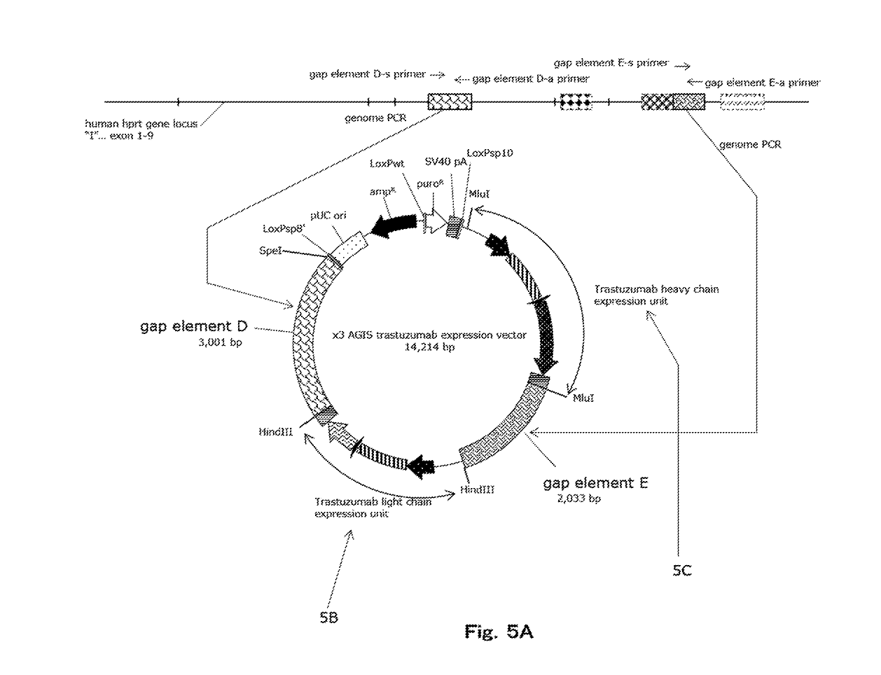

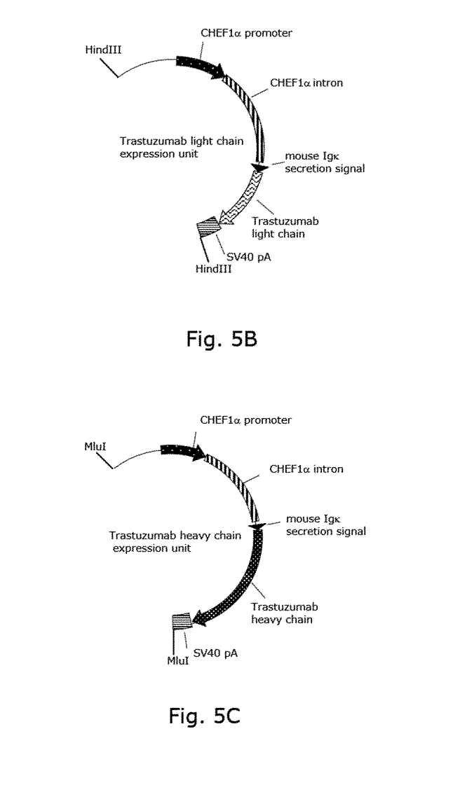

Gap element E (SEQ ID NO: 36) was inserted between the Mlul and HindIII sites of the x3 basic vector obtained above. Then, the gap element D was inserted between HindIII and Spel sites, and subsequently the fragment of the trastuzumab heavy chain expression unit of Step (1-1) was inserted in the Mlul site. Finally, the fragment of the trastuzumab light chain expression unit of Step (1-2) was inserted in the HindIII site to prepare the vector 3 shown in FIG. 5A.

### Step 2: Accumulative Integration of Accumulative Integration Vector into CHO hprt Gene Locus, the Vector Containing Gap Elements Having Mutually Different Sequences Between Expression Units

### Step (2-1) Preparation of CHO hprt AGIS Origin Vector

Using genomic DNA of HEK293 as a template, hEF1 promoter sequence was amplified with an hEF1-s/hEF1-a primer set. To the 5′ terminal of the sense primer was attached the recognition sequence (underlined part) for restriction endonuclease Mlul, and to the 5′ terminal of the antisense primer was attached a phosphate group (P).

hEF1-s primer:
(SEQ ID NO: 37)
5′-ACGCGTagccgaacctctcgcaagtc-3′
hEF1-a primer:
(SEQ ID NO: 38)
5′P-TTTGGCTTTTAGGGGTAGTTTTCACGACAC-3′

Next, using as a template a vector containing EGFP gene sequence possessed by TOTO Ltd. and SV40 poly A sequence, EGFP gene sequence+SV40 poly A sequence was amplified with an EGFP-s/EGFP-a primer set. To the 5′ terminal of the sense primer were attached a phosphate group (P) and LoxPwt sequence (lowercase letters) of Table 1, and to the 5′terminal of the antisense primer were attached the recognition sequence (underlined part) for restriction endonuclease Xbal and the complimentary chain (lowercase letters) for LoxPsp8 sequence of Table 1.

EGFP-s primer:
(SEQ ID NO: 39)
5′P-ATGataacttcgtatagcatacattatacgaagttatCCATGG
TGAGCAGGGCGAGGA-3′
EGFP-a primer:
(SEQ ID NO: 40)
5′-TCTAGAtaccgttcgtataggatactttatacgaagttatTACC
ACATTTGTAGAGGTTT-3′

Then, the hEF1 promoter sequence and the PCR amplification product of EGFP gene sequence+SV40 poly A sequence were ligated to prepare a fragment of hEF1 promoter+EGFP+SV40 poly A.

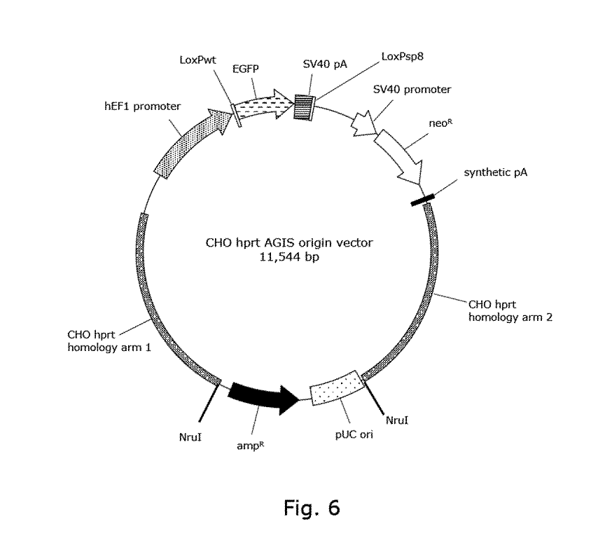

Subsequently, the CHO 2.5kbx2 heavy chain vector described in WO 09/022656 was digested by restriction endonucleases Mlul and Xbal, a fragment of 3.1 kbp was removed, and the hEF1 promoter+EGFP+SV40 poly A fragment prepared in the preceding paragraph was inserted to prepare the CHO hprt AGIS origin vector shown in FIG. 6.

### Step (2-2) Preparation of Cre Expression Plasmid

Using 705-Cre cm, Expression Vector (Funakoshi Co., Ltd.) as a template, a PCR reaction was performed with a Cre-s/Cre-a primer set to amplify Cre recombinase gene. To the 5′ terminal of sense primer was attached the recognition sequence (underlined part) for restriction endonuclease Kpnl, and to the 5′terminal of the antisense primer was attached the recognition sequence (underlined part) for restriction endonuclease BgIII (underlined part).

Cre-s primer:
(SEQ ID NO: 41)
5′-GGTACCGAAGCCGCTAGCGCTACCGGTCGCCACCatgtccaatt
tactgccgtac-3′
Cre-a primer:
(SEQ ID NO: 42)
5′-AGATCTctaatcgccatcttccagca-3′

Next, pmaxGFP plasmid was digested by restriction endonucleases Kpnl and BgIII, the GFP sequence was removed, and the amplification product of the Cre recombinase gene was inserted to prepare a Cre expression plasmid.

### Step (2-3) Integration of CHO hprt AGIS Origin Vector into CHO hprt Gene Locus

A cell line was prepared by adaptation CHO-K1 (ATCC CCL-61) to serum-free suspension culture. This cell line was cultured in AMEM medium (composition: Advanced MEM (Life Technologies), 5% (v/v) FBS (Life Technologies), and 1×GlutaMAX (Life Technologies)) under conditions of 37° C. and 8% CO₂ for 2 days.

The cultured cells were detached with Trypsin/EDTA (Life Technologies) and, after termination of the reaction with AMEM medium, were centrifuged. The cell pellet was washed with PBS (Wako Pure Chemical Industries, Ltd.), thereafter centrifuged, and suspended with PBS. The cell suspension in PBS was subjected to cell density measurement and 2×10⁶ cells were centrifuged. The pellet obtained was suspended in Nucleofection T solution (amaxa) and, thereafter, mixed with 2 μg of linearized CHO hprt AGIS origin vector. To the mixture obtained was applied a voltage by Nucleofectorll (amaxa) program U-023.

The cells to which a voltage had been applied were suspended in 5 mL of AMEM medium, seeded on a 96-well microplate under serial dilution, and cultured under conditions of 37° C. and 8% CO₂ After 2 days from transfection, thereto was added G418 (Life Technologies) (final concentration: 500 μg/mL). After 4 more days, G418 (final concentration: 500 μg/mL) and 6-TG (final concentration: 50 μM) (Wako Pure Chemical Industries, Ltd.) were added and the cells were cultured for further 6 days.

After culture, all wells were checked and G418/6TG resistant colonies were separated. The clones separated were cultured in the AMEM medium to which G418 and 6TG had been added, and the culture was expanded from a 96-well plate to a 24-well palate, then to a 6-well plate, and finally to a 100 mm dish.

Next, each clone was released with Trypsin/EDTA and, after termination of the reaction with the AMEM medium, centrifuged. The cell pellet obtained was washed with CD OptiCHO medium (composition: CD OptiCHO (Life Technologies) and 1×GlutaMax (Life Technologies)), thereafter centrifuged again, and suspended in the CD OptiCHO medium. The cell suspension was subcultured in a 125 ml flask under conditions of 37° C., 8% CO₂, and 128 rpm.

Genomic DNA of a positive clone obtained was extracted with MAG Extractor (TOYOBO). The genomic DNA extracted was analyzed by genomic PCR using a primer set of primer 1/primer 2 and a primer set of primer 3/primer 4.

primer 1:
(SEQ ID NO: 43)
5′-GACACATGCAGACAGAACAG-3′
primer 2:
(SEQ ID NO: 44)
5′-AACTTCTCGGGGACTGTGGG-3′
primer 3:
(SEQ ID NO: 45)
5′-CTATCGCCTTCTTGACGAGT-3′
primer 4:
(SEQ ID NO: 46)
5′-GTTTGCTAACACCCCTTCTC-3′

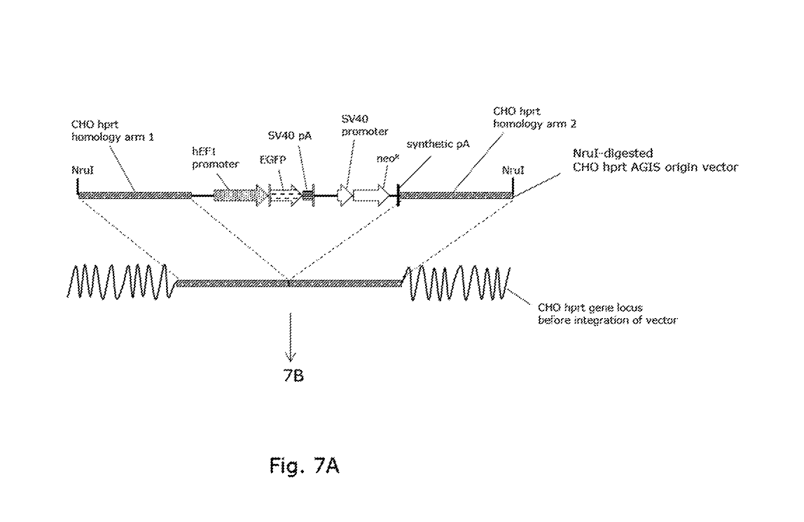

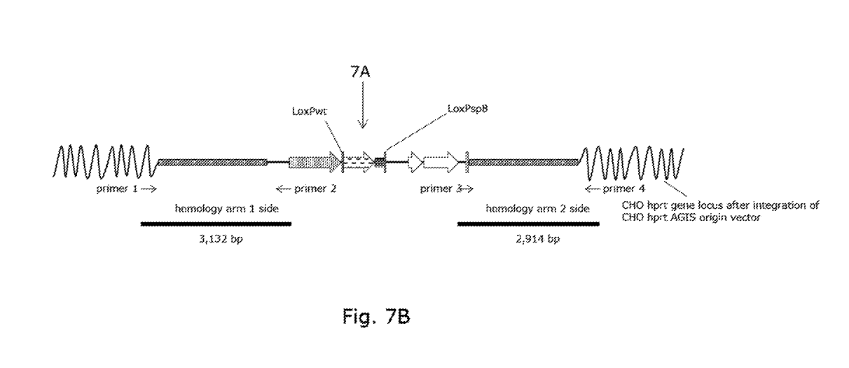

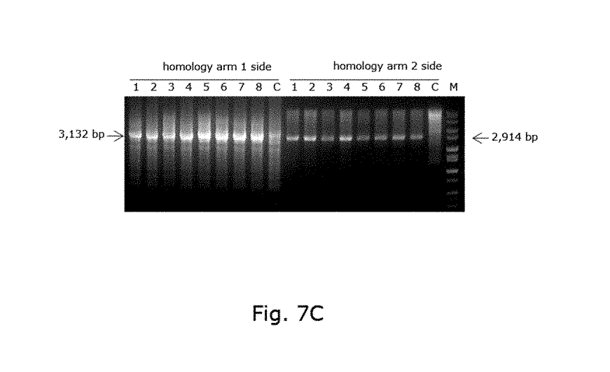

As shown in FIG. 7B, in the case of a correct homologous recombinant clone, a PCR product of 3132 bp is amplified on the homology arm 1 side. Further, a PCR product of 2914 bp is amplified on the homology arm 2 side. The result of analysis was as shown in FIG. 7C. In all of 8 clones analyzed, the amplification fragment of 3132 bp was amplified on the homology arm 1 side and the amplification fragment of 2914 bp was amplified on the homology arm 2 side. Thus, clone No. 1 was used in the following experiment as a cell line having the CHO hprt AGIS origin vector integrated therein.

### Step (2-4) Integration of Vector 1 into CHO hprt Gene Locus

Into the cell line having the CHO hprt AGIS origin vector integrated therein, prepared in Step (2-3), 2 μg of vector 1 and 0.2 μg of Cre expression plasmid were introduced by electroporation as in Step (2-3), and the cell line was seeded on a 96-well plate.

To exchange culture medium after 2 days and 6 days from seeding, and for expanded culturing, there was used AMEM medium+puromycin (final concentration: 12 μg/mL) (Life Technologies).

Genomic DNA of a positive clone obtained was extracted with MAG Extractor. The genomic DNA extracted was analyzed by genomic PCR using a primer set of primer 5/primer 6 and a primer set of primer 7/primer 8.

primer 5:
(SEQ ID NO: 47)
5′-GGGAGCTCAAAATGGAGGAC-3′
primer 6:
(SEQ ID NO: 48)
5′-GAAGAGTTCTTGCAGCTCGG-3′
primer 7:
(SEQ ID NO: 49)
5′-GCTTGCATTGTATGTCTGGC-3′
primer 8:
(SEQ ID NO: 50)
5′-GGTTCTTTCCGCCTCAGAAG-3

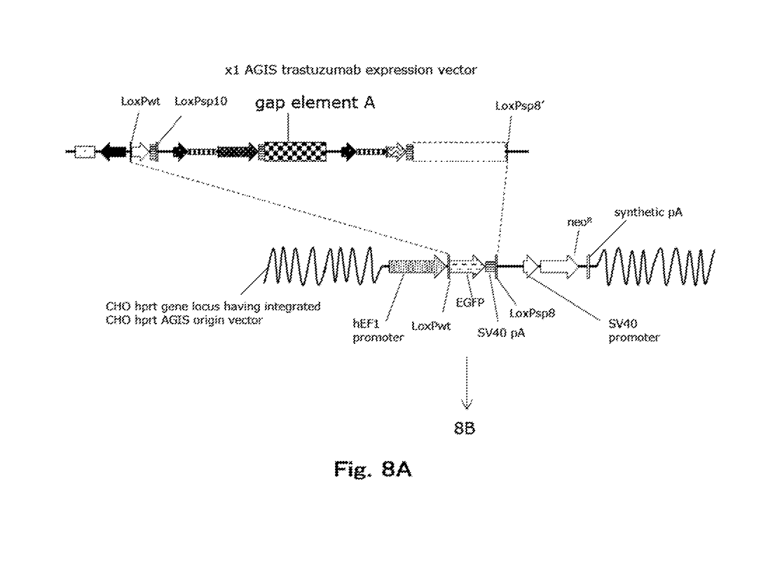

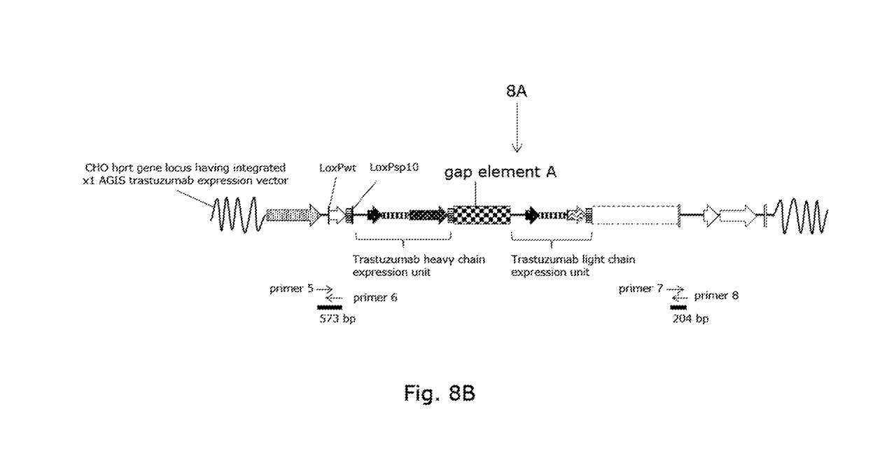

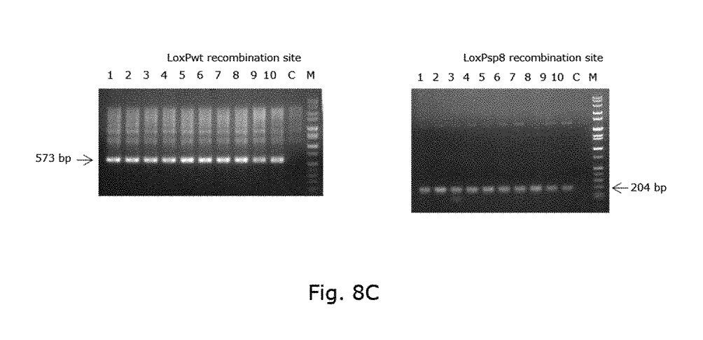

As shown in FIG. 8B, in the case of a correct accumulatively integrated clone, a PCR product of 573 bp is amplified in the vicinity of LoxPwt recombination site. Further, a PCR product of 204 bp is amplified in the vicinity of LoxPst8 recombination site. The result of analysis was as shown in FIG. 8C. In 10 clones out of 10 clones analyzed, the amplification fragment of 573 bp was amplified in the vicinity of LoxPwt recombination site and the amplification fragment of 204 bp was amplified in the vicinity of LoxPst8 recombination site. Thus, clone No. 6 was used in the following experiment as a cell line having the vector 1 integrated therein.

### Step (2-5) Accumulative Integration of Vector 2 into Vector 1-Integrated Cell Line

### Preparation of Cell Example 1

Into the cell line having the vector 1 integrated therein, prepared in Step (2-4), 2 μg of vector 2 and 0.2 μg of Cre expression plasmid were introduced by electroporation as in Step (2-3), and the cell line was seeded on a 96-well plate.

To exchange culture medium after 2 days and 6 days from seeding, and for expanded culturing, there was used AMEM medium+Hygromycin (final concentration: 500 μg/mL) (Life Technologies).

Genomic DNA of a positive clone obtained was extracted with MAG Extractor. The genomic DNA extracted was analyzed by genomic PCR using a primer set of primer 9/primer 10 and a primer set of primer 11/primer 12.

primer 9:
(SEQ ID NO: 51)
5′-GGGAGCTCAAAATGGAGGAC-3′
primer 10:
(SEQ ID NO: 52)
5′-GCAATAGGTCAGGCTCTCGC-3′
primer 11:
(SEQ ID NO: 53)
5′-GAATGGGTTGGCGGAGCTAC-3′
primer 12:
(SEQ ID NO: 54)
5′-GCTGTGGTTCTGATTGTGTGG-3′

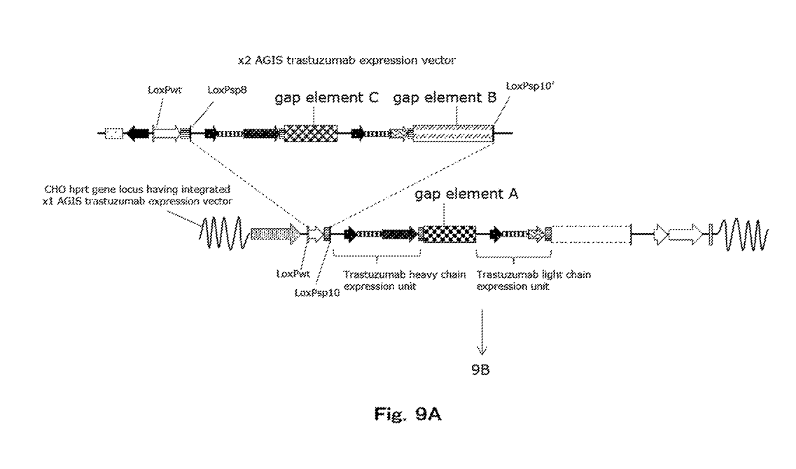

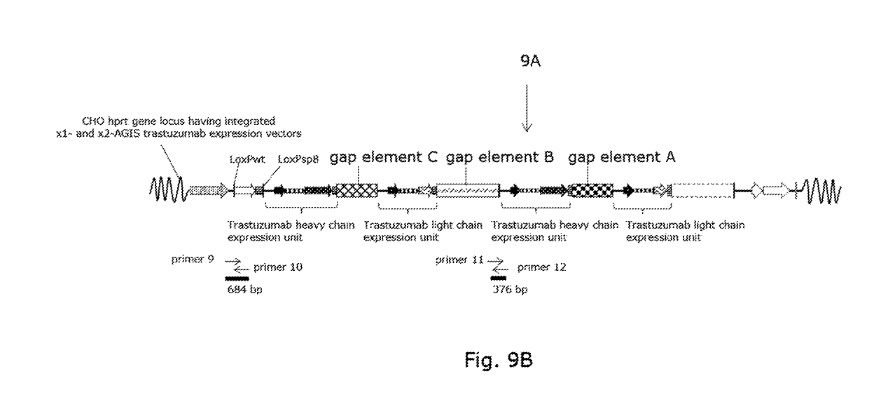

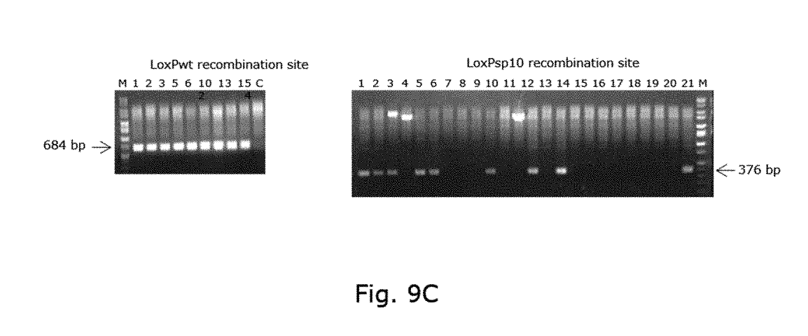

As shown in FIG. 9B, in the case of a correct accumulatively integrated clone, a PCR product of 684 bp is amplified in the vicinity of LoxPwt recombination site. Further, a PCR product of 376 bp is amplified in the vicinity of LoxPsp10 recombination site. The result of analysis was as shown in FIG. 9C. When analysis of the vicinity of LoxPsp10 was performed, in 10 clones out of 24 clones, the amplification product of 376 bp was amplified. When the 10 clones were analyzed in the vicinity of LoxPwt recombination site, in 10 clones out of the 10 clones, the amplification product of 684 bp was amplified. Thus, clone No. 1 was used in the following experiment as a cell line (Cell Example 1) having the vector 1 and vector 2 integrated therein.

### Step (2-6) Accumulative Integration of Vector 3 into Vector 1- and Vector 2-Integrated Cell Line

### Preparation of Cell Example 2

Into the cell line having the vector 1 and vector 2 integrated therein, prepared in Step (2-4), 2 μg of vector 3 and 0.2 μg of Cre expression plasmid were introduced by electroporation as in Step (2-4), and the cell line was seeded on a 96-well plate.

To exchange culture medium after 2 days and 6 days from seeding, and for expanded culturing, there was used AMEM medium+Blasticidin (final concentration: 12 μg/mL) (Life Technologies).

Genomic DNA of a positive clone obtained was extracted with MAG Extractor. The genomic DNA extracted was analyzed by genomic PCR using a primer set of primer 13/primer 14 and a primer set of primer 15/primer 16.

primer 13:
(SEQ ID NO: 55)
5′-GGGAGCTCAAAATGGAGGAC-3′
primer 14:
(SEQ ID NO: 56)
5′-GCTGTGGTTCTGATTGTGTGG-3′
primer 15:
(SEQ ID NO: 57)
5′-GACTACAAGTGCGTGCCATC-3′
primer 16:
(SEQ ID NO: 58)
5′-GCTGTGGTTCTGATTGTGTGG-3′

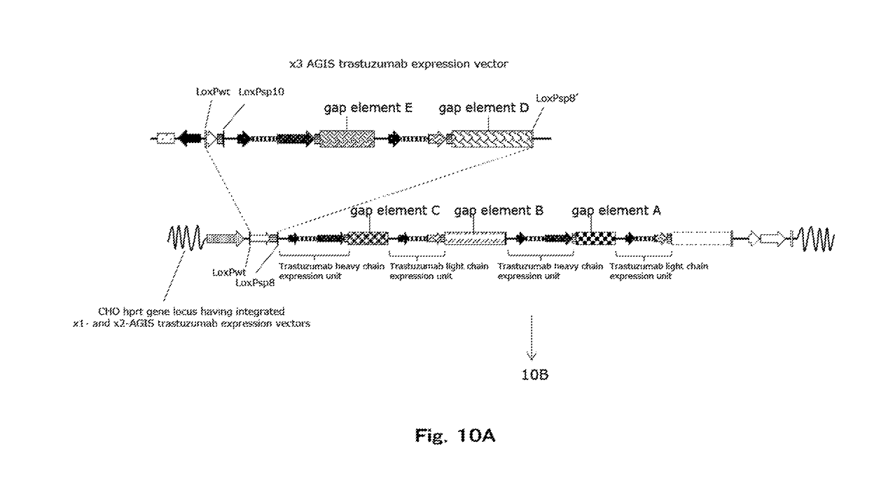

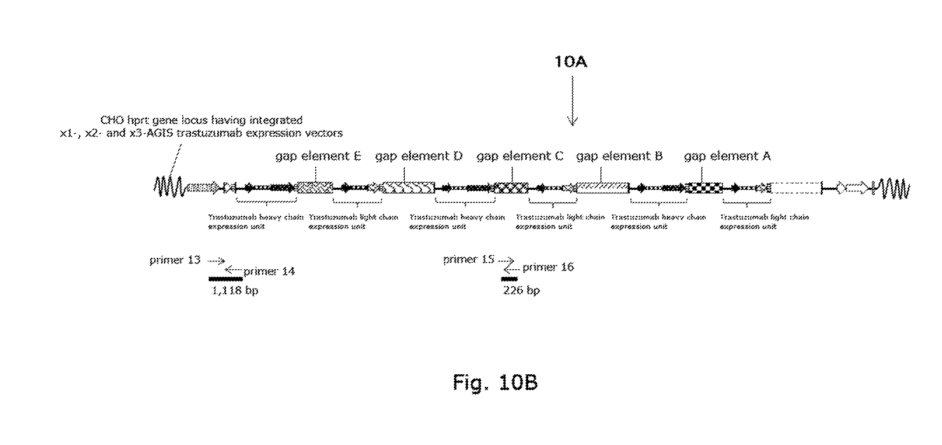

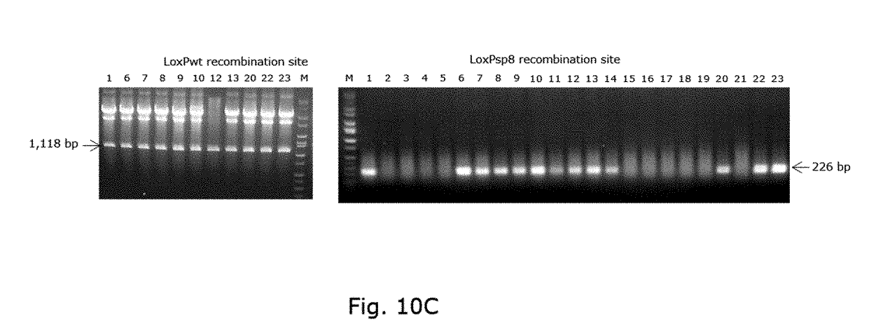

As shown in FIG. 10B, in the case of a correct accumulatively integrated cell, a PCR product of 1118 bp is amplified in the vicinity of LoxPwt recombination site. Further, a PCR product of 226 bp is amplified in the vicinity of LoxPsp8 recombination site. The result of analysis was as shown in FIG. 10C. When analysis of the vicinity of LoxPsp8 was performed, in 11clones out of 23 clones, the amplification product of 226 bp was amplified. When the 11 clones were analyzed in the vicinity of LoxPwt recombination site, in 11 clones out of the 11 clones, the amplification product of 1118 bp was amplified. Thus, clone No. 22 was used in the following evaluation as a cell line (Cell Example 2) having the vector 1, vector 2, and vector 3 integrated therein.

### Evaluation: Evaluation of Accumulative Integration Vector-Integrated CHO, the Vector Containing Gap Elements Having Mutually Different Sequences Introduced Among Expression Units

### Evaluation 1: Measurement of Homology of Gap Elements

Homology between gap elements of the vector 1, vector 2, and vector 3, prepared in Steps (1-3), (1-4), and (1-5), respectively, was measured as follows.

Electronic files of the gap elements were read into GENETYX Ver. 10 software, and “Nucleotide vs Nucleotide Homology” of the “Homology” command was selected.

Next, in the “Parameter” dialogue, checks were entered into both of “Original Sequence” and “Complement Sequence,” and the values of “Unit Size” and “Scores alignments” were respectively set to “6” and “100”.

Then, electronic files of the gap elements to be compared were added to “Target Sequence” in the “Sequence” dialogue, and “OK” was selected for execution.

Of the values of “Identity (%),” the output measurement results, the highest value was employed as the value of homology.

As a result, homology between respective gap elements was 51% for gap element A-gap element B, 45% for gap element A-gap element C, 48% for gap element A-gap element D, 45% for gap element A-gap element E, 44% for gap element B-gap element C, 52% for gap element B-gap element D, 46% for gap element B-gap element E, 44% for gap element C-gap element D, 45% for gap element C-gap element E, and 45% for gap element D-gap element E.

### Evaluation 2: Expression Level Analysis of Trastuzumab Heavy Chain and Light Chain in Vector 1-Integrated Cell Line (Comparative Example 1), Vector 1- and Vector 2-Integrated Cell Line (Example 1), and Vector 1-, Vector 2-, and Vector 3-Integrated Cell Line

### Example 2

Genomic DNAs of the vector 1-integrated cell line (hereinafter called “Comparative Example 1”), the vector 1- and vector 2-integrated cell line (hereinafter called “Example 1”), and the vector 1-, vector 2-, and vector 3-integrated cell line (hereinafter called Example 2) were extracted with MAG Extractor, the cell lines having been prepared in Steps (2-4), (2-5), and (2-6). The copy numbers of trastuzumab heavy chain and light chain in the extracted genomic DNAs were quantified by real-time PCR.

Quantification of the copy number of the trastuzumab heavy chain was performed with a primer set of heavy chain forward primer/heavy chain reverse primer, and a heavy chain TaqMAN probe.

heavy chain forward primer:
(SEQ ID NO: 59)
5′-ACGAGGACCCTGAAGTGAAGTTC-3′
heavy chain reverse primer:
(SEQ ID NO: 60)
5′-TGTTCCTCTCTAGGCTTGGTCTTG-3′
heavy chain TaqMAN probe:
(SEQ ID NO: 61)
5′-TACGTGGACGGCGTGGA-3′

Quantification of the copy number of the trastuzumab light chain was performed with a primer set of light chain forward primer/light chain reverse primer, and a light chain TaqMAN probe.

light chain forward primer:
(SEQ ID NO: 62)
5′-TCTACTCCGCCTCCTTCCTGTA-3′
light chain reverse primer:
(SEQ ID NO: 63)
5′-TGGCAGTAGTAGGTGGCGAAGT-3′
light chain TaqMAN probe:
(SEQ ID NO: 64)
5′-ATCCGGCACCGACTT-3′

As a real-time PCR instrument, there was used 7300 Real-Time PCR (Applied Biosystems). The quantified values were standardized by the copy number of CHO endogenous GAPDH.

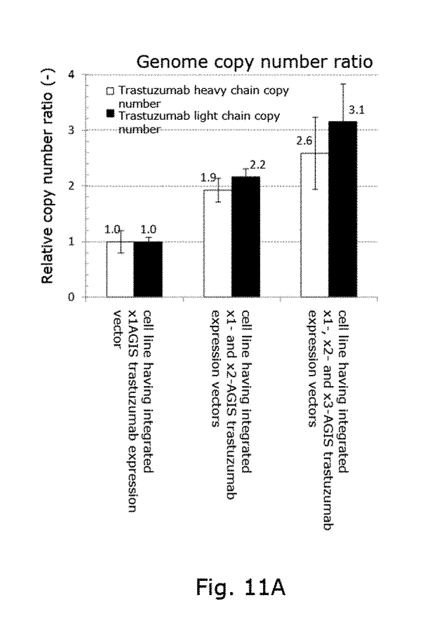

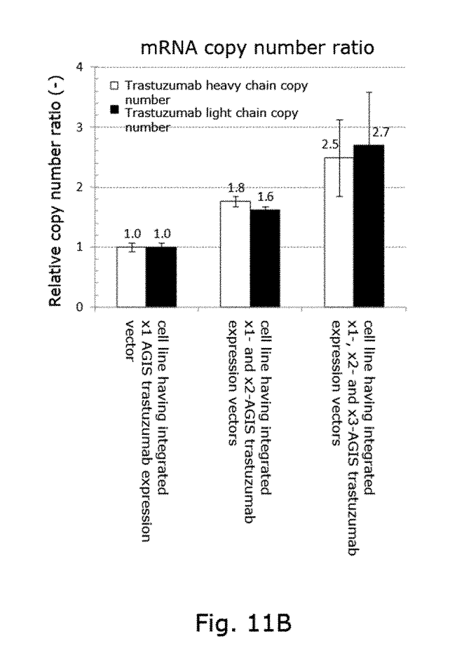

The quantification results were as shown in FIG. 11A. The copy numbers of the trastuzumab heavy chain gene and the trastuzumab light chain gene were in agreement with the copy number of each introduced.

Next, the cells of Comparative Example 1, Example 1, and Example 2 were adjusted in the CD OptiCHO medium to 3×10⁵ cells/mL and were cultured in a 125 mL flask for 2 days under conditions of 37° C., 8% CO₂, and 128 rpm.

Culture solutions of the previous paragraph were centrifuged, the cell pellets were washed with PBS (TaKaRa), mRNA was extracted from 5×10⁴ cells with TaqMan Gene Expression Cells-to-CT Kit (Life Technologies), and cDNA was synthesized by using the Kit. Copy numbers of trastuzumab heavy chain and trastuzumab light chain in the prepared cDNA were quantified by using a primer set of heavy chain forward primer/heavy chain reverse primer, and a heavy chain TaqMAN probe. The quantified values were standardized by the copy number of mRNA CHO endogenous GAPDH, and the relative copy number was calculated by setting that of a cell line to 1.0, in which cell line the copy number of trastuzumab gene introduced was 1.

The quantification results were as shown in FIG. 11B. The relative copy numbers of trastuzumab heavy chain mRNA were 1.0, 1.8, and 2.5 respectively in cell lines in which copy numbers of introduced trastuzumab heavy chain genes were 1, 2, and 3, and increased nearly in proportion to the copy number of genes.

Also, as shown in FIG. 11B, the relative copy numbers of trastuzumab light chain mRNA were 1.0, 1.6, and 2.7 respectively in cell lines in which copy numbers of introduced trastuzumab light chain genes were 1, 2, and 3, and increased nearly in proportion to the copy number of genes.

### Evaluation 2: Analysis of Trastuzumab Productivity and Long-Term Expression Stability of Cells of Comparative Example 1, Example 1, and Example 2

The cells of Comparative Example 1, Example 1, and Example 2 were each maintained by subculture in a 125 mL flask under conditions of the CD OptiCHO medium, 37° C., 8% CO₂, and 128 rpm, and an amount of trastuzumab produced by each cell line was timely measured.

Measurement of the amount of trastuzumab produced was performed as follows. The cell line immediately after establishment or in the course of subculture were adjusted in the CD OptiCHO medium to 2×10⁵ cells/mL and were cultured in a 125 mL flask for 8 days under conditions of 37° C., 8% CO₂, and 128 rpm. The cell density of culture solution on the 8th day of culture was measured, and the culture solution was centrifuged to obtain a culture supernatant. The amount of trastuzumab in the culture supernatant was measured by using Biosensor/Protein A Tray (ForteBIO) on the Blitz system (ForteBIO).

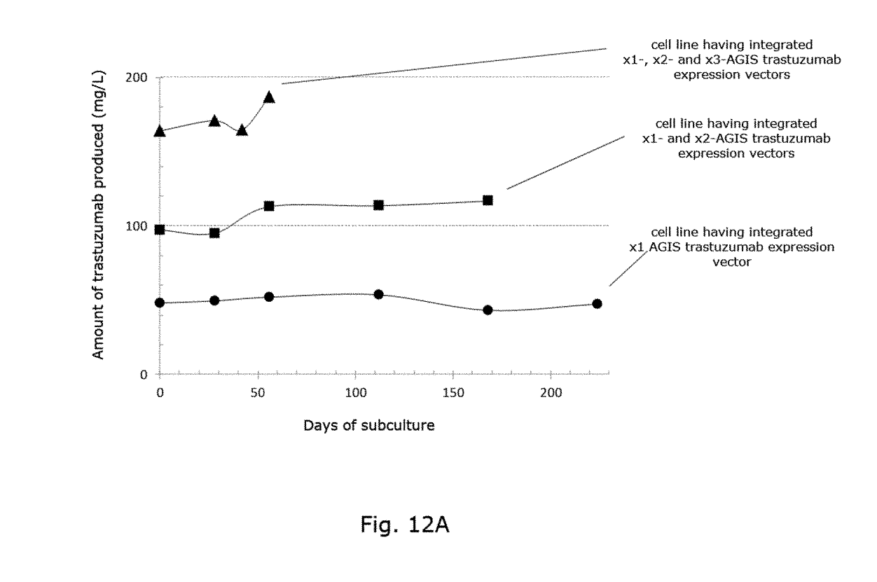

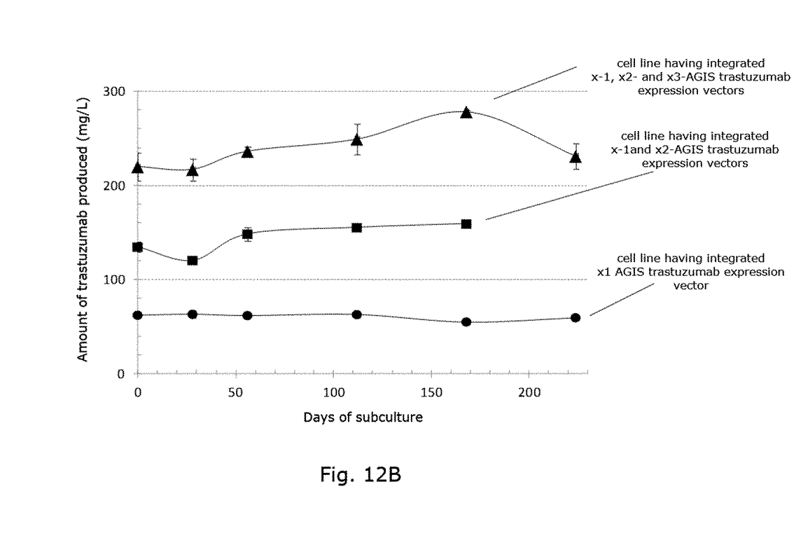

The expression level of trastuzumab in batch culture of Comparative Example 1 was 48.0 mg/L immediately after establishment. Further, during subculture for 224 days, the expression level was stably maintained (FIG. 12A).

The expression level of trastuzumab in batch culture of Example 1 was 97.1 mg/L immediately after establishment. The expression level increased to 113 mg/L by 56 days of subculture, and thereafter was stably maintained at 116 mg/L during 168 days of subculture (FIG. 12A).

The expression level of trastuzumab in batch culture of Example 2 was 164 mg/L immediately after establishment. Further, during subculture for 56 days, the expression level was stably maintained (FIG. 12A).

In the cells of Comparative Example 1, Example 1, and Example 2, the expression levels of trastuzumab relative to the copy number of introduced trastuzumab gene showed a strong positive linear correlation. Further, there occurred no decrease in the expression level due to subculture, and long-term, stable expression was confirmed (FIG. 12A).

Furthermore, the cells of Example 2 were maintained by subculture and, in the same manner as above, the amounts of trastuzumab produced after subculture for 112 days, 168 days, and 224 days were measured, and compared with the amounts in the cells of Comparative Example 1 and Example 1 on these days. Thereat, in order to reduce the quantification error due to day to day variation as much as possible, the culture supernatants which had been stored in the above tests, the results of which are shown in FIG. 12A, were subjected to remeasurement of expression levels of trastuzumab therein. That is, with regard to Comparative Example 1, the culture supernatants sampled immediately after establishment and 28 days, 56 days, 112 days, 168 days, and 224 days thereafter, and stored at −80° C. were subjected simultaneously to remeasurement of the expression level of trastuzumab. Similarly, with regard to Example 1, the culture supernatants sampled immediately after establishment and 28 days, 56 days, 112 days, 168 days, and 224 days thereafter, and stored at −80° C. were subjected simultaneously to remeasurement of the expression level of trastuzumab. Furthermore, with regard to Example 2, the expression levels of trastuzumab before the above-mentioned days were also remeasured simultaneously in consideration of the quantification error, using the culture supernatants sampled immediately after establishment and 28 days and 56 days thereafter, and stored at −80° C.

As a result of remeasurement, the expression level of trastuzumab in batch culture of Comparative Example 1 was 62.3 mg/L. In addition, the expression level was stably maintained after subculture of 224 days (FIG. 12B).

As a result of remeasurement, the expression level of trastuzumab in batch culture of Example 1 was 135 mg/L immediately after establishment. The expression level increased to 148 mg/L by 56 days of subculture and, thereafter, was stably maintained at 159 mg/L during subculture of 168 days (FIG. 12B).

As a result of remeasurement, the expression level of trastuzumab in batch culture of Example 2 was 220 mg/L immediately after establishment. In addition, the expression level was stably maintained during subculture of 224 days (FIG. 12B).

### Comparative Example 2

### Example of Cell in Which Homology Between Mutually Different Gap Elements is 100%

A “x2 AGIS gap element A vector,” which has as a gap element the same sequence as the full length of gap element A, was prepared as follows.

A sequence of gap element A′ (SEQ ID NO: 65) was obtained by conducting artificial gene synthesis. In this sequence, the recognition sequence (AAGCTT) for restriction endonuclease HindIII is attached to the 5′ terminal, and the recognition sequence (ACTAGT) for restriction endonuclease Spel is attached to the 3′ terminal. Further, the sequence of 2043 bp just before the Spel at 3′ terminal is completely the same as the sequence contained in 2051 bp of the gap element A of Step (1-3). On the other hand, 1 kbp after HindIII recognition sequence at 5′ terminal is the same as the gap element B of Step (1-4).

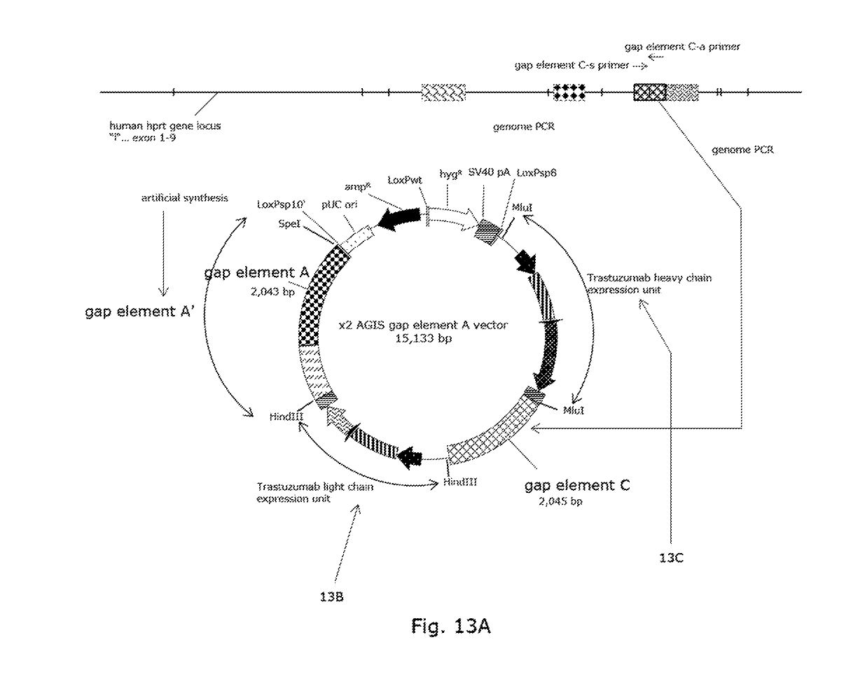

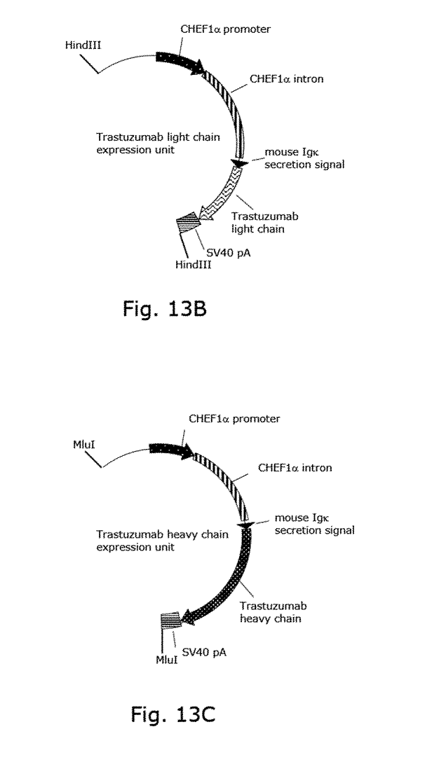

The gap element C was inserted between the M1ul and HindIII sites of the x2 basic vector obtained in Step (1-4). Then, the gap element A was inserted between the HindIII and Spel sites, and subsequently the fragment of the trastuzumab heavy chain expression unit of Step (1-1) was inserted in the M1ul site. Finally, the fragment of the trastuzumab light chain expression unit of Step (1-2) was inserted in the HindIII site to prepare the x2 AGIS gap element A vector shown in FIG. 13A.

Next, the x2 AGIS gap element A vector was accumulatively integrated into the vector 1-integrated cell line as follows to prepare the cells.

The x2 AGIS gap element A vector was integrated into the vector 1-integrated cell line, prepared in Step (2-4), in the same manner as in Step (2-3) to obtain a positive clone.

Genomic DNA of the positive clone obtained was extracted with MAG Extractor. The genomic DNA extracted was analyzed by genomic PCR using a primer set of primer 9/primer 10 and a primer set of primer 16/primer 12.

primer16:
(SEQ ID NO: 66)
5′-TCCCAAAGTACCGGGATTAC-3′

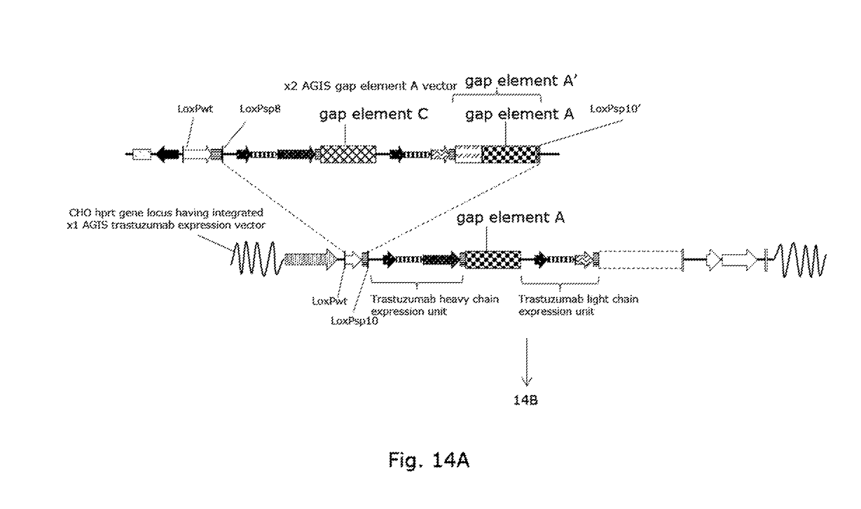

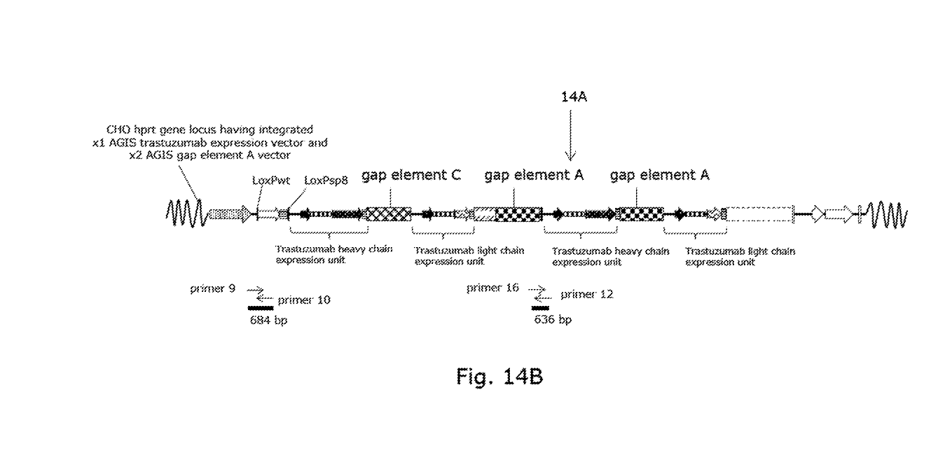

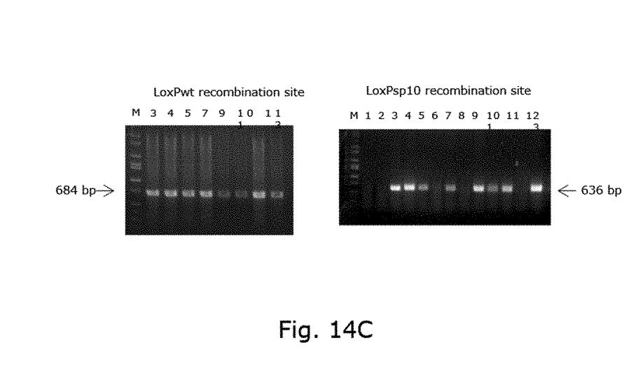

As shown in FIG. 14B, in the case of a correct accumulatively integrated clone, a PCR product of 684 bp is amplified in the vicinity of LoxPwt recombination site. Further, a PCR product of 636 bp is amplified in the vicinity of LoxPsp10 recombination site. The result of analysis was as shown in FIG. 14C. When analysis of the vicinity of LoxPsp10 recombination site was performed, in 8 clones out of 13 clones, the product of 636 bp was amplified. When the 8 clones were analyzed in the vicinity of LoxPwt recombination site, in 8 clones out of 10 clones, the amplification product of 684 bp was amplified.

Clone No. 3 was selected and, in the same manner as in Evaluation 1, the copy numbers of the heavy chain and light chain of trastuzumab in the genomic DNA was quantified by real-time PCR in the same manner as in Evaluation 1.

As a result, the copy number of trastuzumab heavy chain was 1.9±0.1 and the copy number of light chain was 1.9±0.1, which were approximately in agreement with 2, the number of genes introduced. Thus, clone No. 3 was used in the following evaluation as vector 1- and x2 AGIS gap element A vector-introduced cell line (hereinafter, this cell is called “Comparative Example 2”).

### Evaluation: Evaluation of Vector-Integrated CHO Cell Line, the Vector Having Gap Elements of Mutually Homologous Sequences Introduced Between Expression Units

### Evaluation 3: Analysis of Expression Levels of Trastuzumab Heavy Chain and Light Chain in Cells of Comparative Example 2

The cells of Comparative Example 2 were maintained by subculture in the same manner as in Evaluation 2, and the production amount of trastuzumabin the cell line was timely measured.

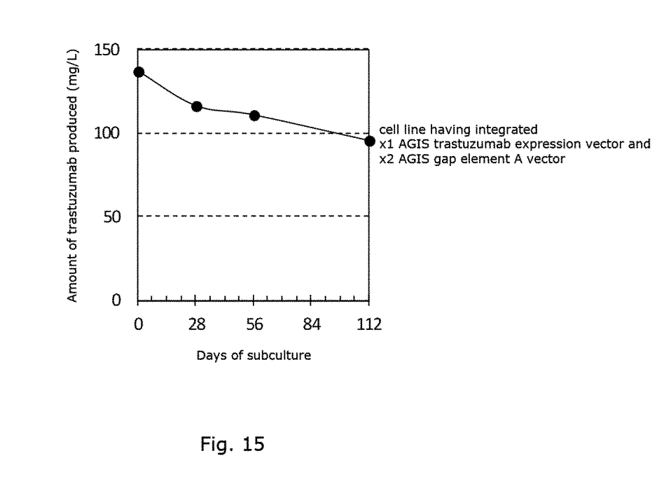

The expression level of trastuzumab in batch culture of Comparative Example 2 was 137 mg/L immediately after establishment. The expression level decreased with continuation of subculture, and after 112 days of subculture, the expression level decreased to 95.5 mg/L (FIG. 15).

### Example 3

### Example of Cell Having One Gap Element of 0.5 kbp (#1) Example of Cell in Which Gap Element B of Example 1 is Changed to Chain Length of 0.5 kbp

A “x2 AGIS Gap Element B 500 Vector” having one gap element of 541 bp was prepared as follows.

Using genomic DNA of HEK293 as a template, a gap element B500 (SEQ ID NO: 67) of 541 bp was PCR amplified with a gap element B500-s/gap element B-a primer set, and the length of amplification product was confirmed by agarose gel electrophoresis. To the 5′ terminal of the sense primer was attached the recognition sequence (underlined part) for restriction endonuclease HindIII.

gap element B500-s primer:
(SEQ ID NO: 68)
5′-AAGCTTaatctttgtcagcagttccc-3′

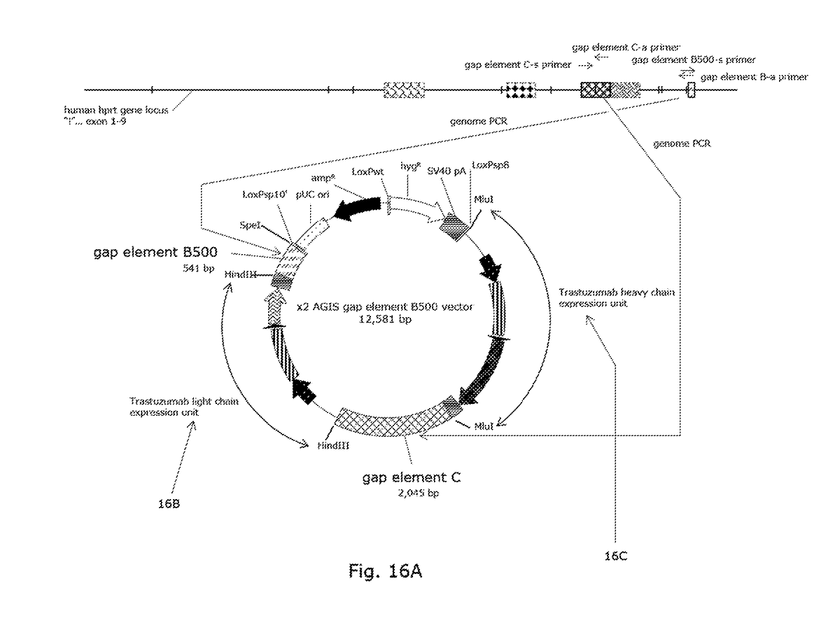

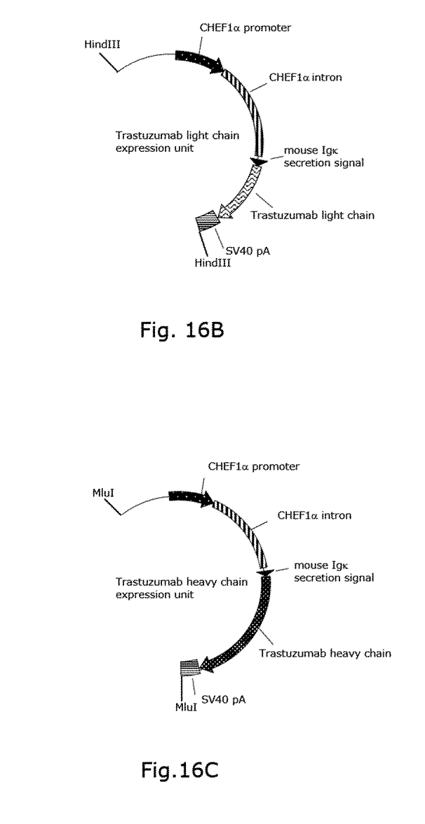

The gap element C was inserted between the M1ul and HindIII sites of the x2 basic vector prepared in Step (1-4). Then, the gap element B500 was inserted between the HindIII and Spel sites, and subsequently the fragment of the trastuzumab heavy chain expression unit of Step (1-1) was inserted in the M1ul site. Finally, the fragment of the trastuzumab light chain expression unit of Step (1-2) was inserted in the HindIII site to prepare the x2 AGIS gap element B500 vector shown in FIG. 16A.

Next, the x2 AGIS gap element B500 vector was accumulatively integrated into the vector 1-integrated cell line as follows to prepare the cells.

The x2 AGIS gap element B500 vector was integrated into the vector 1-integrated cell line, prepared in Step (2-4), in the same manner as in Step (2-3) to obtain a positive clone.

Genomic DNA of the positive clone obtained was extracted with MAG Extractor. The genomic DNA extracted was analyzed by genomic PCR using a primer set of primer 9/primer 10 and a primer set of primer 11/primer 12.

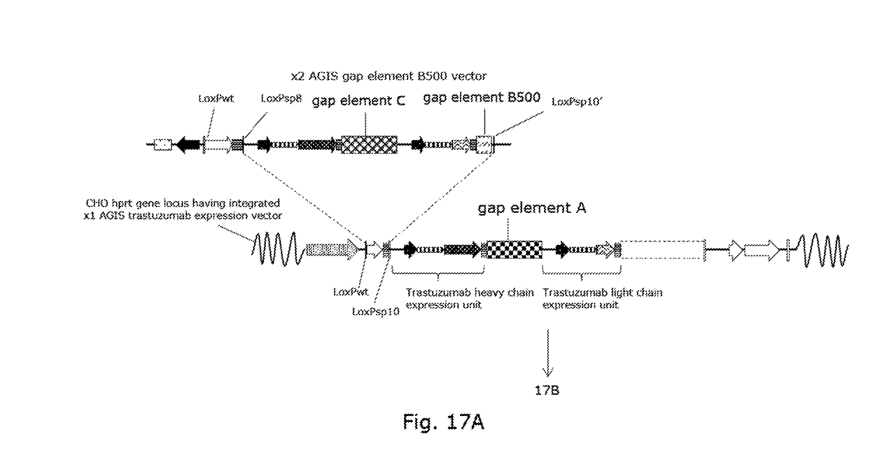

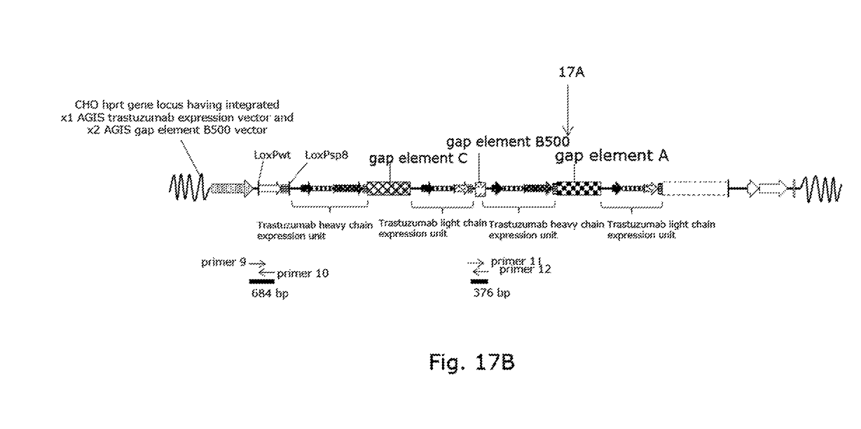

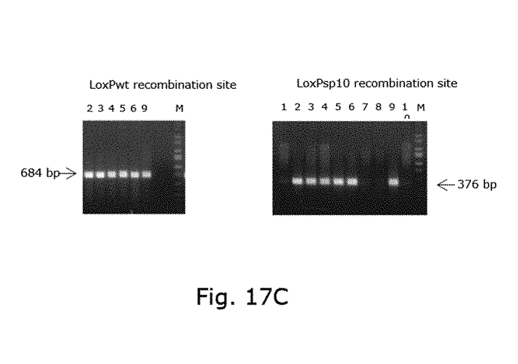

As shown in FIG. 17B, in the case of a correct accumulatively integrated clone, a PCR product of 684 bp is amplified in the vicinity of LoxPwt recombination site. Further, a PCR product of 376 bp is amplified in the vicinity of LoxPsp10 recombination site. The result of analysis was as shown in FIG. 17C. When analysis of the vicinity of LoxPsp10 recombination site was performed, in 6 clones out of 10 clones, the amplification product of 376 bp was amplified. When the 6 clones were analyzed in the vicinity of LoxPwt recombination site, in 6 clones out of the 6 clones, the amplification product of 684 bp was amplified.

Clone No. 2 was selected and, in the same manner as in Evaluation 1, the copy numbers of the heavy chain and light chain of trastuzumab in the genomic DNA was quantified by real-time PCR.

As a result, the copy number of trastuzumab heavy chain was 1.9±0.1 and the copy number of light chain was 2.0±0.2, which were approximately in agreement with 2, the number of genes introduced. Thus, clone No. 2 was used in the following evaluation as vector 1- and x2 AGIS gap element B500 vector-introduced cell line (hereinafter, this cell is called “Example 3”).

### Evaluation: Evaluation of Vector-Integrated CHO Cell Line, the Vector Having One Gap Element of 0.5 kbp (541 bp)

### Evaluation 4: Analysis of Expression Levels of Trastuzumab Heavy Chain and Light Chain in Cells of Example 3

The cells of Example 3 were maintained by subculture in the same manner as in Evaluation 2, and the production amount of trastuzumab in the cell line was timely measured.

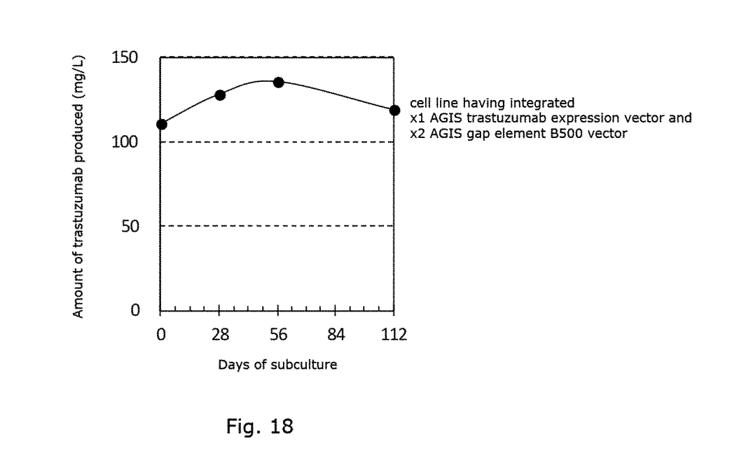

The expression level of trastuzumab in batch culture of Example 3 was 110 mg/L immediately after establishment. The expression level was 119 mg/L after 112 days of subculture, and thus the expression level was stably maintained (FIG. 18).

### Example 4

### Example of Cell Having One Gap Element of 0.5 kbp (#2) Example of Cell in Which Gap Element C of Example 1 is Changed to Chain Length of 0.5 kbp

A “x2 AGIS Gap Element C500 Vector” having one gap element of 493 bp was prepared as follows.

Using genomic DNA of HEK293 as a template, a gap element C500 (SEQ ID NO: 69) of 493 bp was PCR amplified with a gap element C500-s/gap element C-a primer set, and the length of amplification product was confirmed by agarose gel electrophoresis. The recognition sequence (underlined part) for restriction endonuclease HindIII was attached to the 5′ terminal of the sense primer.

gap element C500-s primer:
(SEQ ID NO: 70)
5′-AAGCTTTCATCTCTCAACAACTCTGG-3′

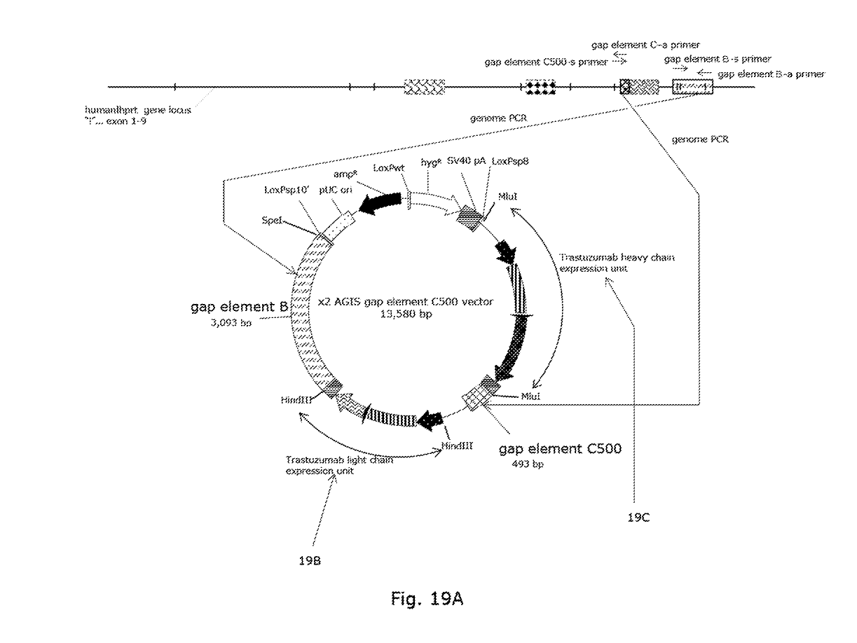

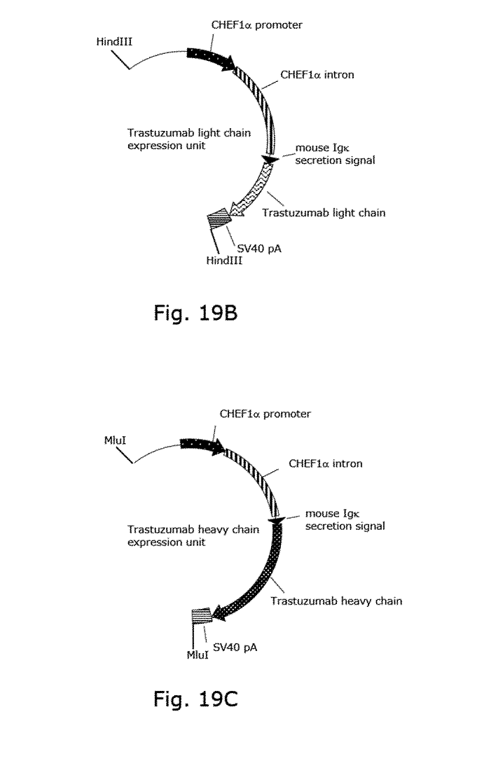

The gap element C500 was inserted between the M1ul and HindIII sites of the x2 basic vector prepared in Step (1-4). Then, the gap element B was inserted between the HindIII and Spel sites, and subsequently the fragment of the trastuzumab heavy chain expression unit of Step (1-1) was inserted in the M1ul site. Finally, the fragment of the trastuzumab light chain expression unit of Step (1-2) was inserted in the HindIII site to prepare the x2 AGIS gap element C500 vector shown in FIG. 19A.

Next, the x2 AGIS gap element C500 vector was accumulatively integrated into the vector 1-integrated cell line as follows to prepare the cells.

The x2 AGIS gap element C500 vector was integrated into the vector 1-integrated cell line, prepared in Step (2-4), in the same manner as in Step (2-3) to obtain a positive clone.

Genomic DNA of the positive clone obtained was extracted with MAG Extractor. The genomic DNA extracted was analyzed by genomic PCR using a primer set of primer 9/primer 10 and a primer set of primer 11/primer 12.

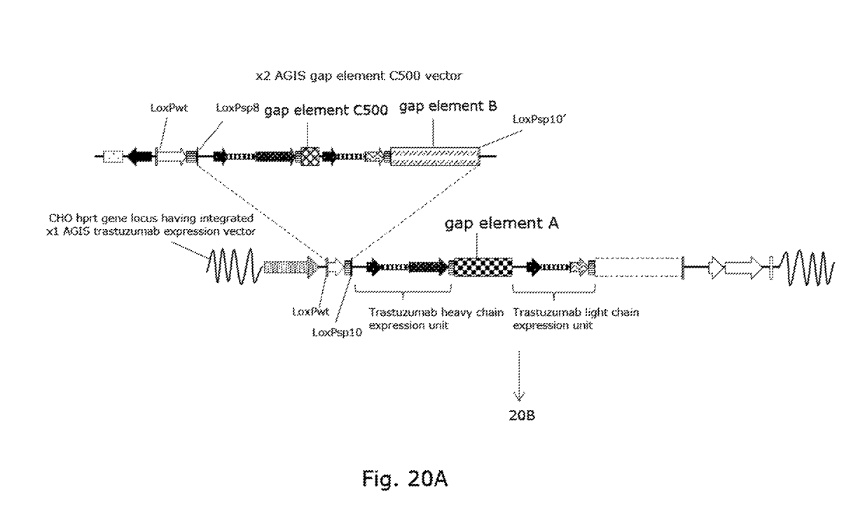

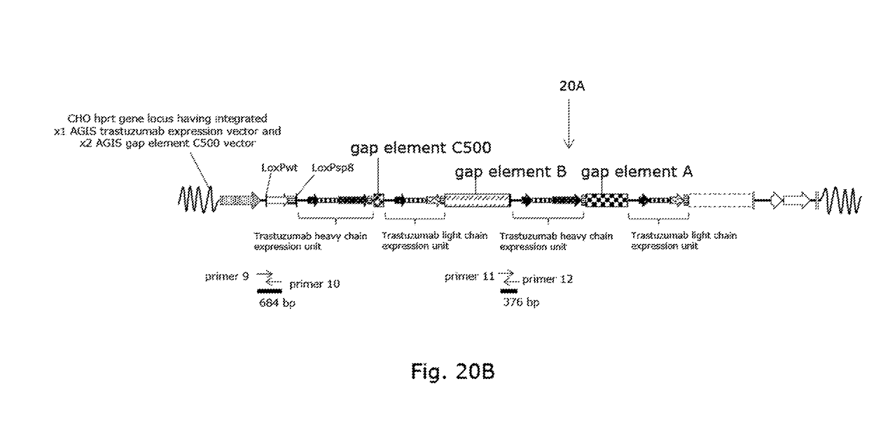

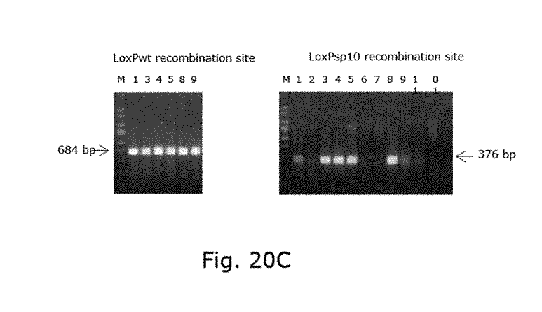

As shown in FIG. 20B, in the case of a correct accumulatively integrated clone, a PCR product of 684 bp is amplified in the vicinity of LoxPwt recombination site. Further, a PCR product of 376 bp is amplified in the vicinity of LoxPsp10 recombination site. The result of analysis was as shown in FIG. 20C. When analysis of the vicinity of LoxPsp10 recombination site was performed, in 6 clones out of 11 clones, the amplification product of 376 bp was amplified. When the 6 clones were analyzed in the vicinity of LoxPwt recombination site, in 6 clones out of the 6 clones, the amplification product of 684 bp was amplified.

Clone No. 5 was selected and, in the same manner as in Evaluation 1, the copy numbers of the heavy chain and light chain of trastuzumab in the genomic DNA was quantified by real-time PCR.

As a result, the copy number of trastuzumab heavy chain was 2.0±0.4 and the copy number of trastuzumab light chain was 1.8±0.5, which were approximately in agreement with 2, the number of genes introduced. Thus, clone No. 5 was used in the following evaluation as vector 1- and x2 AGIS gap element C500 vector-integrated cell line (hereinafter, this cell is called “Example 4”).

### Evaluation: Evaluation of Vector-Integrated CHO Cell Line, the Vector Having One Gap Element of 0.5 kbp (493bp)

### Evaluation 5: Analysis of Expression Levels of Trastuzumab Heavy Chain and Light Chain in Cells of Example 4

The cells of Example 4 were maintained by subculture in the same manner as in Evaluation 2, and the production amount of trastuzumab in the cell line was timely measured.

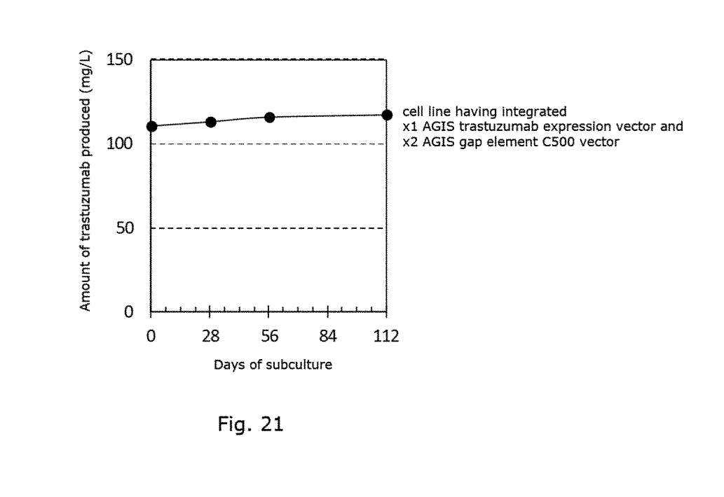

The expression level of trastuzumab in batch culture of Example 4 was 111 mg/L immediately after establishment. The expression level was 118 mg/L after 102 days of subculture, and thus the expression level was stably maintained (FIG. 21).

### Example 5

### Example of Cell in Which Gap Elements A, B, and C of Example 1 are All Changed to Chain Length of 0.5 kbp

A “x1 AGIS Gap Element A500 Vector” having one gap element of 502 bp was prepared as follows.

Using genomic DNA of HEK293 as a template, a gap element A500 (SEQ ID NO: 70) of 502 bp was PCR amplified with a primer set of gap element A500-s/gap element A-a, and the length of amplification product was confirmed by agarose gel electrophoresis. To the 5′ terminal of the sense primer was attached the recognition sequence (underlined part) for restriction endonuclease HindIII.

gap element A500-s primer:
(SEQ ID NO: 71)
5′-AAGCTTGGGGGTATTTTTGTGTGCAG-3′

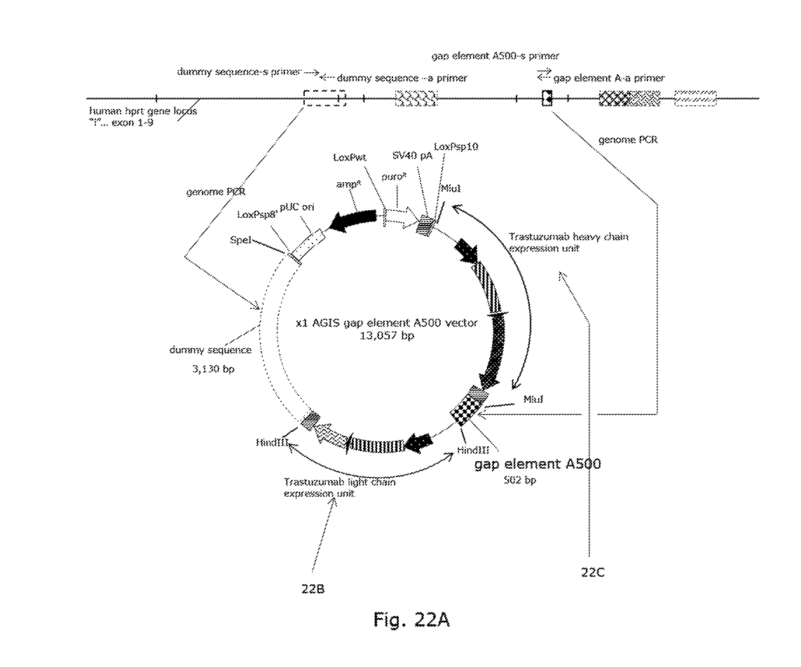

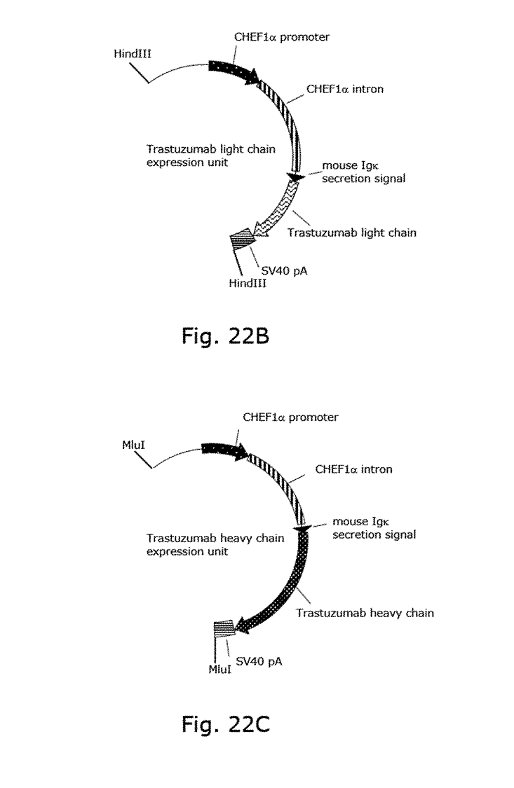

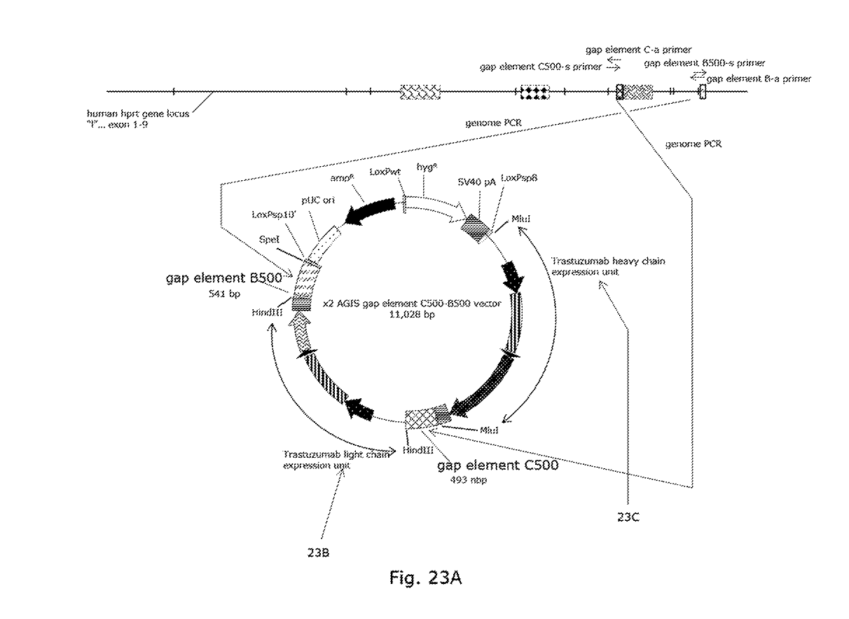

The gap element A500 was inserted between the M1ul and HindIII sites of the x1 basic vector prepared in Step (1-3). Then, a dummy sequence was inserted between the HindIII and Spel sites, and subsequently the fragment of the trastuzumab heavy chain expression unit of Step (1-1) was inserted in the M1ul site. Finally, the fragment of the trastuzumab light chain expression unit of Step (1-2) was inserted in the HindIII site to prepare the x1 AGIS gap element A500 vector shown in FIG. 22A.

Next, a “x2 AGIS gap element C500-B500 vector” having two gap elements of 493 bp and 541 bp, respectively, was prepared as follows.

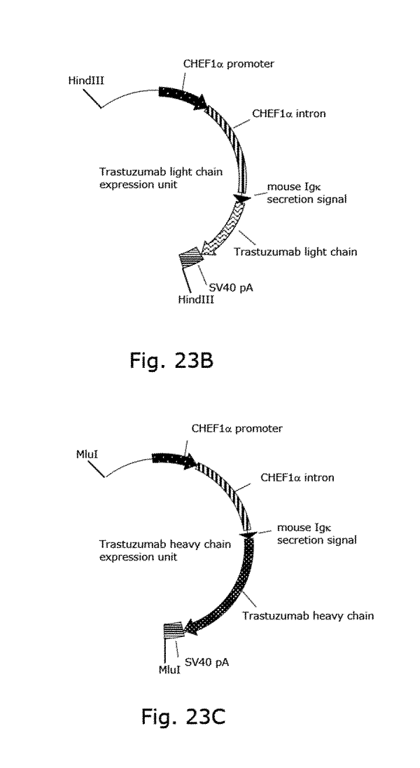

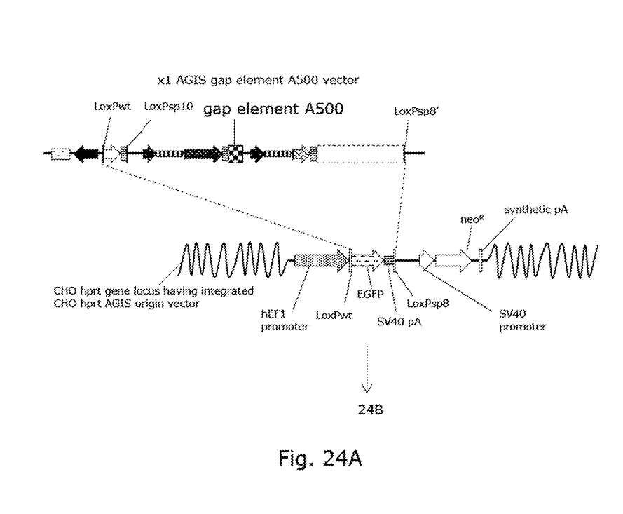

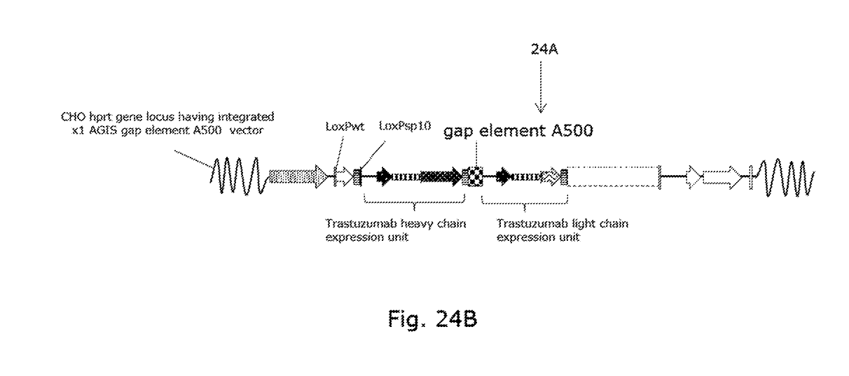

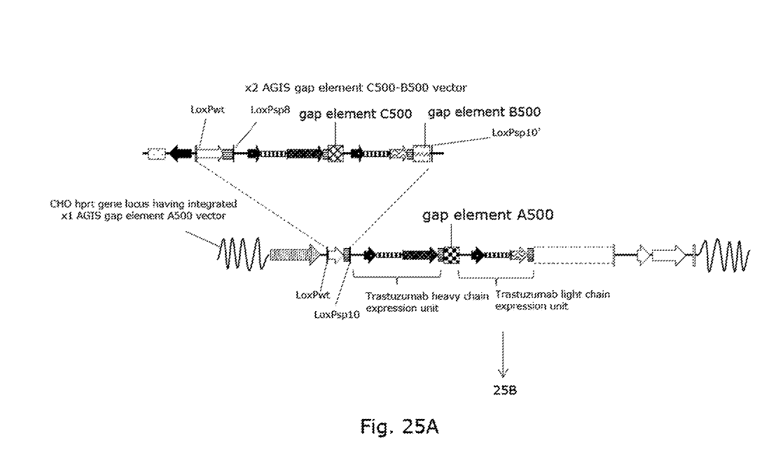

The gap element C500 of Step (5-1) was inserted between the M1ul and HindIII sites of the x2 basic vector prepared in Step (1-4). Then, the gap element B500 of Step (4-1) was inserted between the HindIII and Spel sites, and subsequently the fragment of the trastuzumab heavy chain expression unit of Step (1-1) was inserted in the M1ul site. Finally, the fragment of the trastuzumab light chain expression unit of Step (1-2) was inserted in the HindIII site to prepare the x2 AGIS gap element C500-B500 vector shown in FIG. 23A.

Next, the x1 AGIS gap element A500 vector was accumulatively integrated into the CHO hprt AGIS origin vector-integrated cell line as follows.

Into the CHO hprt AGIS origin vector-integrated cell line, prepared in process (2-3), 2 μg of x1 AGIS gap element A500 vector and 0.2 μg of Cre expression plasmid were introduced by voltage application in the same manner as in Step (2-4), and the cell line was seeded on a 96-well plate.

To exchange culture medium after 2 days and 6 days from seeding, and for cell expansion, there was used AMEM medium+puromycin (final concentration: 12 μg/mL).

The cells which showed proliferation in the well were collected to obtain x1 AGIS gap element A500 vector-integrated cells.

Then, into the x1 AGIS gap element A500 vector-integrated cell line, the x2 AGIS gap element C500-B500 vector was accumulatively integrated as follows to prepare the cells of Example 5.

Into the x1 AGIS gap element A500 vector-integrated cell line prepared in Step (6-3), the x2 AGIS gap element C500-B500 vector was integrated in the same manner as in Step (2-3) to obtain a positive clone.

Genomic DNA of the positive clone obtained was extracted with MAG Extractor. The genomic DNA extracted was analyzed by genomic PCR using a primer set of primer 7/primer 8, a primer set of primer 9/primer 10, and a primer set of primer 11/primer 12.

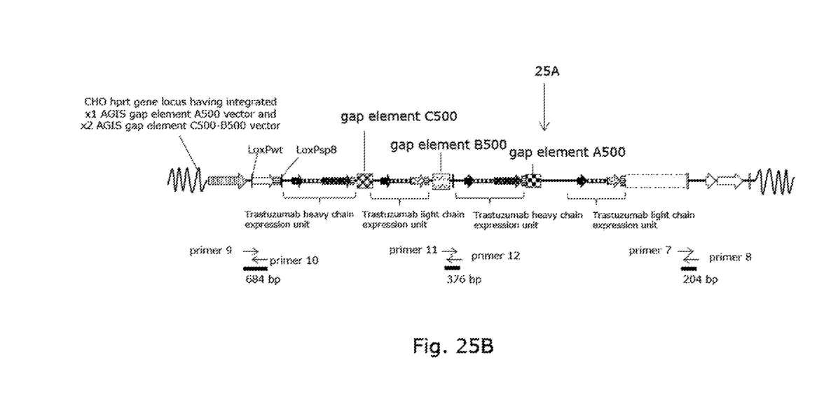

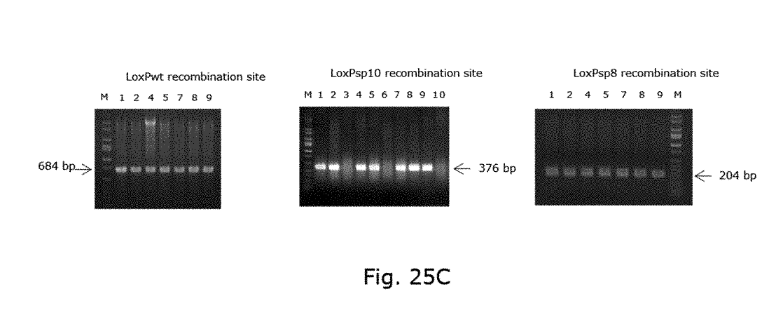

As shown in FIG. 25B, in the case of a correct accumulatively integrated clone, a PCR product of 684 bp is amplified in the vicinity of LoxPwt recombination site. Further, a PCR product of 376 bp is amplified in the vicinity of LoxPsp10 recombination site. Furthermore, a PCR product of 204 bp is amplified in the vicinity of LoxPsp8 recombination site. The result of analysis was as shown in FIG. 25C. When analysis of the vicinity of LoxPsp10 was performed, in 7 clones out of 10 clones, the product of 376 bp was amplified. When the 7 clones were analyzed in the vicinity of LoxPwt recombination site, in 7 clones out of the 7 clones, the amplification product of 684 bp was amplified. Also, when the 7 clones were analyzed in the vicinity of LoxPsp8 recombination site, in 7 clones out of the 7 clones, the amplification product of 204 bp was amplified.

Clone No. 2 was selected and, in the same manner as in Evaluation 1, the copy numbers of the heavy chain and light chain of trastuzumab in the genomic DNA were quantified by real-time PCR.

As a result, the copy number of trastuzumab heavy chain was 1.8±0.1 and the copy number of light chain was 2.0±0.3, which were approximately in agreement with 2, the number of genes introduced. Thus, clone No. 2 was used in the following evaluation as x1 AGIS gap element A500 vector and x2 AGIS gap element C500-B500 vector-introduced cell line (hereinafter, this cell is called “Example 5”).

### Evaluation: Evaluation of Vector-Integrated CHO Cell Line in Which All of 3 Gap Elements are 0.5 kbp

### Evaluation 6: Analysis of Expression Levels of Trastuzumab Heavy Chain and Light Chain in Cells of Example 5

The cells of Example 5 were maintained by subculture in the same manner as in Evaluation 2, and the production amount of trastuzumab in the cell line was timely measured.

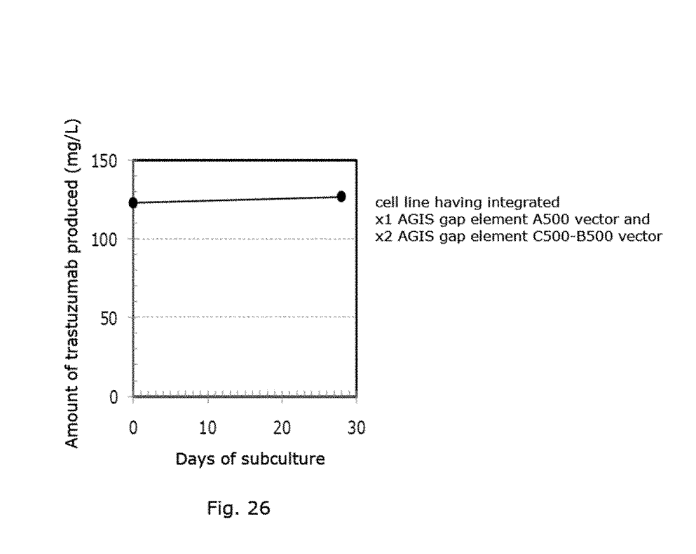

The expression level of trastuzumab in batch culture of Example 5 was 123 mg/L immediately after establishment. The expression level was 127 mg/L after 28 days of subculture, and thus was stably maintained (FIG. 26).

### [Sequence Listing]

What is claimed is:

1. A mammalian cell comprising multiple expression units which comprise an exogenous gene of interest and which are integrated therein, wherein the number of said multiple expression units is 3 or more; and further comprising exogenous gap elements which are provided among said multiple expression units wherein said gap elements have chain lengths of 0.5 kbp or more and 20 kbp or less, and said gap elements are mutually different sequences.

2. The cell according to claim 1, wherein the chain length of said gap elements is in the range form 1 kbp or more to 20 kbp or less.

3. The cell according to claim 1, wherein one or more of said gap elements have chain lengths of 1 kbp or more.

4. The cell according to claim 1, wherein multiple kinds of said genes of interest are integrated.

5. The cell according to claim 1, wherein said multiple expression units and said gap elements are introduced into inside of said animal cell.

6. The cell according to claim 5, wherein said multiple expression units and said gap elements are introduced into a chromosome of said animal cell.

7. The cell according to claim 5, wherein a site of said chromosome of said animal cell where said multiple expression units and said gap elements are introduced is a stable expression gene locus.

8. The cell according to claim 7, wherein said gene locus is a hypoxanthine-guanine phosphoribosyltransferase (hprt) gene locus.

9. The cell according to claim 1, wherein homology between said gap elements adjacent to each other is either of 55% or less or 50% or less.

10. The cell according to claim 1, wherein homology between said mutually different gap elements is either of 55% or less or 50% or less.

11. The cell according to claim 1, wherein said mammalian cell is a CHO cell.

12. The cell according to claim 1, wherein said gene of interest is a gene which encodes a protein.

13. The cell according to claim 12, wherein said protein is an antibody.

14. A method for producing a substance of interest, comprising a step of culturing said cell according to claim 1.

15. A method for producing the cell according to claim 1, wherein said multiple expression units and said gap elements are introduced by using accumulative gene integration system (AGIS).


SEQUENCE LISTING (71 sequences)

SEQ ID NO: 1 - protein, 21 aa, Unknown
  Signal peptide
METDTLLLWVLLLWVPGSTGD

SEQ ID NO: 2 - DNA, 26 bp, Artificial Sequence
  Primer
ctcgagctcgatgagtttggacaaac

SEQ ID NO: 3 - DNA, 49 bp, Artificial Sequence
  Primer
atcgatgtcgacaagctttctagaacgcgttaccacatttgtagaggtt

SEQ ID NO: 4 - DNA, 49 bp, Artificial Sequence
  Primer
atcgatgtcgacaagctttctagaacgcgtccacacaatcagaaccaca

SEQ ID NO: 5 - DNA, 26 bp, Artificial Sequence
  Primer
gaattcgcggtggttttcacaacacc

SEQ ID NO: 6 - DNA, 26 bp, Artificial Sequence
  Primer
atcgatgcttacatggcgatagctag

SEQ ID NO: 7 - DNA, 26 bp, Artificial Sequence
  Primer
atcgatgaatcaggggataacgcagg

SEQ ID NO: 8 - DNA, 60 bp, Artificial Sequence
  Primer
actagtctaccgttcgtataaagtatcctatacgaagttattagcttcagtgagcaaaag

SEQ ID NO: 9 - DNA, 26 bp, Artificial Sequence
  Primer
ggatcctcgcgtaggtggcacttttc

SEQ ID NO: 10 - DNA, 61 bp, Artificial Sequence
  Primer
ggatccataacttcgtatagcatacattatacgaagttatccaccgagtcaagcccacggt

SEQ ID NO: 11 - DNA, 72 bp, Artificial Sequence
  Primer
actagtaagcttacgcgttaccgttcgtataattatggttatacgaagttatacgatcataatcagccatac

SEQ ID NO: 12 - DNA, 26 bp, Artificial Sequence
  Primer
aagcttggcaacatagcgagacttcg

SEQ ID NO: 13 - DNA, 26 bp, Artificial Sequence
  Primer
actagtgtgtacagaaggctgaaagg

SEQ ID NO: 14 - DNA, 2051 bp, Artificial Sequence
  Gap element
tgaaagtgtgagttccggagcctgacaactgattcaaagcatggcttagtacttcctaactctgaccttgggcaagttacttaacctctctgtgtcccatatgtgattagggtgaggttgataatagcagccatagagttaagaggattaagtgctataatgcaagtagagctcttacaacagtttctggtaaatcactcaataaattcagacatactattattttaagaaatctcaaagagttttcttgtaccttaaaattctcctagtgtgaaccattggttttggtatattgtgcttccatgtagtttaatatcaagatgtttttagatttcccttttaatttatttgttgacccattggttgttcaggagcatgctgtttacctgaaaataatggagatattaaggtatttgaatatttatcttctagtacattgaaaaactttttgagagtaaccaataataaatgatggaatgctactgctttttttttttgaagctgccagttattgtttacttacactatgccaaatataaaggcattaatctcataaaagtttcacaacaatcctgtgagggagacgatatccccattttacaaatcaggaaattaagacttaataaggttaaaagacttgcccaaagtcacagaaccagtaagtggtagagcttgaatttgaatacagacctgactctaaagctcttttctttctttagattttagtgttcattgcttacttgaatgagtatctataagaaaactttaacatgtaaaacttctgtgaaattatcttgtcccatatcagggtcatgtcaaactaatgtcctcctcagcatctttggaaaacttcagaggagaaatgagctttgcccctcctgttcatttcatataccactgttagacctgtccttccctttcagcatgctttgtccatatttagaagctgttgaagccattacttgtctggtcagtttttagtgctggaatggacctagccttttaggccttctgagatttagtttgatctcgtctttcccacctaatggctctgttctactacatagatttgatctgaaacagttctctgtttctaaaataactttcttttcatgatagtcacagtaaagtacatttattatggaaaaatcaataagtataacgagtgaaagttatttcttggtggtaagattatgggattatttgaactttctgtttcattgtattttatttatttatttatttttgtgatggagtctcactctgctgcccaggctggagtgcagtagtacgatcttggctcactgcaacctccccttcccagttcaagtgattctcctgcctcagactcccaagtagctgggattacaggcgcacgccaccatgcctggctaatttttttatctttagtagagacagggtttcaccatgttgaccaggctgatctccaactcctgatctcaggtatccacctgcctcagcctcccaaagtaccgggattacgggtgtgagccaccctgcctggcctcattttgtcttttgggggtatttttgtgtgcagatatatatgtatataaatatttttccctcttttccccagttagtatttgagcagatgaactttggacccgaatacctgtattcaagtctctaataccacttcttggctattttcattttatcaaatggcctcttatcctcgtttttctcatttattaagtagagatgtaactacttgatataattcaaaaactcaataatggcattcttttgttttttagactctagtgtctgtactcgttgtaccatgctgggattcatttgaacaattgcatggcttttttagtgtattattaaatttgcagtttacttagaatttactgggacctcatacaaatgggaaaaaaacataactgtgttactcatttgctgtgtgcctttggattgaccctattttttgtattcattttctccccatgtcctgagttccactttgaataaaaagtaatttttttcctgcctgtaaaataggctaccaataggctgcagtt

SEQ ID NO: 15 - DNA, 26 bp, Artificial Sequence
  Primer
acgcgttgaaagtgtgagttccggag

SEQ ID NO: 16 - DNA, 52 bp, Artificial Sequence
  Primer
aagcttaactgcagcctattggtagcacgcgttgaaagtgtgagttccggag

SEQ ID NO: 17 - DNA, 61 bp, Artificial Sequence
  Primer
actagtctaccgttcgtataaccataattatacgaagttattagcttcatgtgagcaaaag

SEQ ID NO: 18 - DNA, 26 bp, Artificial Sequence
  Primer
ggatcctcgcgtaggtggcacttttc

SEQ ID NO: 19 - DNA, 61 bp, Artificial Sequence
  Primer
ggatccataacttcgtatagcatacattatacgaagttatccaaaaagctgaactcaccgc

SEQ ID NO: 20 - DNA, 72 bp, Artificial Sequence
  Primer
actagtaagcttacgcgttaccgttcgtataggatactttatacgaagttattagctcactcattaggcacc

SEQ ID NO: 21 - DNA, 26 bp, Artificial Sequence
  Primer
acgcgtcgttttctcactggctgctg

SEQ ID NO: 22 - DNA, 26 bp, Artificial Sequence
  Primer
aagcttactctcatagtacctgtccc

SEQ ID NO: 23 - DNA, 3093 bp, Artificial Sequence
  Gap element
gctcattgacactctgtacccattaatcctccttattagctccccttcaatggacacatgggtagtcagggtgcaggtctcagaactgtccttcaggttccaggtgatcaaccaagtgccttgtctgtagtgtcaactcattgctgccccttcctagtaatccccataatttagctctccatttcatagtctttccttgggtgtgttaaaagtgaccatggtacactcagcacggatgaaatgaaacagtgtttagaaacgtcagtcttctcttttgtaatgccctgtagtctctctgtatgttatatgtcacattttgtaattaacagcttgctggtgaaaaggaccccacgaagtgttggatataagccagactgtaagtgaattactttttttgtcaatcatttaaccatctttaacctaaaagagttttatgtgaaatggcttataattgcttagagaatatttgtagagaggcacatttgccagtattagatttaaaagtgatgttttctttatctaaatgatgaattatgattctttttagttgttggatttgaaattccagacaagtttgttgtaggatatgcccttgactataatgaatacttcagggatttgaatgtaagtaattgcttctttttctcactcatttttcaaaacacgcataaaaatttaggaaagagaattgttttctccttccagcacctcataatttgaacagactgatggttcccattagtcacataaagctgtagtctagtacagacgtccttagaactggaacctggccaggctagggtgacacttcttgttggctgaaatagttgaacagctttaatatacaataattgttgcattattatttcagatgataaatgtggtcataagtaagaaataaatgatcgagtttagtcttttaattcactgtcctttgaatacctgcctcttactctggaggcagaagtcccatggatgtgtttatgaacatggttgaggaagatttaggaagactgcaacagtacactacctaaagcaggttttttactccatctttttttgccacgtacactggcctcccactttgatatgcttgaaattatctccttgatttgtctttcaaaactacatattgaggctggttgcggtggctcacacctgtaatcctagcactttgggaggccaagccggacagatcacttgaggtcaggagttcgagaccagcctggcaaacatgatgaaaccccacctttactaaaaatacaaaaattagccaggcgtagtggtgtgtgcctgtaacccagctacctgggaggctgaggcaggagaatcactggaacccgggaggcagaggctacagtgagccaacatcacgccactgcactccagcctgggtgacagagcaagactctgtctcaaaacaaaacaaaaaacaaaaaactacgtattaagacaagaaacagactgggcgcggtggctcacgcctgtaatcccagcactttgggaggctgaggcgggcggatcacaaggtcaggagatcgagaccatcctggctaacacggtgaaaccccgtctctactaaaaaatagaaaaaattagctggggtggtggcgggcgcctatagtctcagctactcgggaggctgaggcaggagaatggcgtgaacccgggaggcagagcttgcagtgagcagagatcgtgccactgcactccagtctgggtgacagagcaagactccgtctcaaaaaaaaaaacaaaaacaagaaacaaattaaactaatgtgatagactactgctttgttttcaaaagatacactccccaaaagttactgatctaaaatacagtagtactatctctgtttagtaagaaccctgacaactaatagtgttcttatatgtaaaatgctattcttgcctttcatttcagaatatactttttaaatgtgaatttctggatttttttttatagcatgtttgtgtcattagtgaaactggaaaagcaaaatacaaagcctaagatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaatttaactgctttatgaattcaatttaaaaatccttaaaataagtcctgtctctttaaaaaaacctatgcatagttatcatttctctacaaattaacctagtttagttttctgttggttccattttccttgtttgttaagttttagtagctagtttaattgtaatctcaatgattatgtggtagaatgggttggcggagctacaaaaattcctagctacttcagagacattaaatttcagacacatggtacactttatattacattttactatgctaaaataacacggctttcttttggaattctgttcagtttttcagattgtaatctcagctacatctcaacagattgttctcagatatgtcctattaccttctttgtgtagatagtgctttattgactaagaacaatgacaacaacaccttttgttttctgggaataggagaaaagttttaagccaaaactcttaattgcttatctgctccacgtgag

SEQ ID NO: 24 - DNA, 26 bp, Artificial Sequence
  Primer
aagcttgctcattgacactctgtacc

SEQ ID NO: 25 - DNA, 26 bp, Artificial Sequence
  Primer
actagtctcacgtggagcagataagc

SEQ ID NO: 26 - DNA, 2045 bp, Artificial Sequence
  Gap element
cgttttctcactggctgctggccaggggttgctcccagatatttaaaggctcatgccctagcccatgacagtctcacaacatggcagctgacttcttcaaaaccagcaggagaatcttgctctagtctaccacataacctaatcacaggagcggctatcccgttattttcacagatcctggtcacattcaaggggagggaacccttctgtgtgtgtacaccaggaggcaggaattttttttttctttttcttttttgttaaaaagtcttaaagtcttttatccctaaaggaggcaggaattttgagagccatcagaattctgcctaccacagcccagaaatctgcatttttcacaagtctccagccatgatgtttctgatggctcacactgctttattccatttttaaagagtatttttattgaaaagcattagggttatggtttaaaaaatattttccctaacaaagatgggtttgtttagagtcctacttttgactaaatagctgagattcacttttatgtaaagtcattttatagcgttattaatttgggtgcctttaaaaatagtataaagcatgtttctcgagtgtagtctgttagccacctatattggagagttgggaggagagagtctctatcttgaatttatgggaaaaattctaaaatactttttataatgaaggacaacatcataactccctaataaaatgtgcatgtatatattcaaatttgctgtcattgatcctgcacctacaaaatccagtcctgggggctggcattcttactgcttgctgagggccagatgatatagattccagaatatctccatgtagattttggtgagaattactgtgctgaaaagaatgacagtattgcagttatacatgggggttttggtactttatattgtgactctgaatttaaagctatgcaatgtcttcttttttgaaaggatataattgacactggcaaaacaatgcagactttgctttccttggtcaggcagtataatccaaagatggtcaaggtcgcaaggtatgtatgacattttgacacagaatattttcctcatttgaagggggattaagtgattgcttctttttaaggataaatgttttcaactgtcattttatcttcgaaaagtaatgtaatctcatataagacttaagatataatccttttaaataattttgtcatgtgttaataaagctcataattacagtcacttccttgcctaatattaacatttggtttttcagcatgctaattatatcagtttgtcctgaatagcatggcagaggattttgggcccccttgcaaaattaagaataaggattccaaagcgggtgaggaagtgataggaaggggtgggccctgaagatctggacctcctggaattgagtgatgaatgctgcatcttctttgtgtctgtagtgaaattttataatgcctgcttccttttttattaagtcggcctcacctcctcaccttacctatgctgttttacttttgcttttatagttctacctgtgtttatttctcattttcgtttcatctctcaacaactctggggtggcattattattcccacttttcagataaggttactgaggcatagggaattgtccaaaggtacagagctagtccgctatagagatgagatttgaacccagggaacctggctcacagtttatgcttttgctaccttaagtttttaatagagtgacatcaaacaaacatttaagaatatgtttttcttttccttttataatttcattaaaaacattaagtctctgatcagtctgcagtttttatgtaggggtcaggtaatgttctaacttctgctttttcctaagtgattaacaggtttttataagcccttttgaaaaaatcacggtatctgtcgagcatctttgaatcagagtaagccttctagtgagtcatatgtcagcagtttgactgtatgggcttttctaatatccagttcaagtgtttatcagtgagtttttcttttaaatagatttgggacaggtactatgagagt

SEQ ID NO: 27 - DNA, 61 bp, Artificial Sequence
  Primer
actagtctaccgttcgtataaagtatcctatacgaagttattagcttcatgtgagcaaaag

SEQ ID NO: 28 - DNA, 26 bp, Artificial Sequence
  Primer
ggatcctcgcgtaggtggcacttttc

SEQ ID NO: 29 - DNA, 61 bp, Artificial Sequence
  Primer
ggatccataacttcgtatagcatacattatacgaagttatccgccaagccctgtcccagga

SEQ ID NO: 30 - DNA, 72 bp, Artificial Sequence; identical to SEQ ID NO: 11
  Primer

SEQ ID NO: 31 - DNA, 26 bp, Artificial Sequence
  Primer
acgcgtcaacctactgtggcatagac

SEQ ID NO: 32 - DNA, 26 bp, Artificial Sequence
  Primer
aagcttacaggcagaggttatcctgg

SEQ ID NO: 33 - DNA, 3001 bp, Artificial Sequence
  Gap element
agatcacagctcactgtggccttgaccttctgggctcaagtgattctcccacttcggcctcccgagtagctgggactaacaggtgtgcaccaccatggctggctaatttttttttatgtttgtagagattgggtcttactgtgttgcccaggctgatcccgaactcctgggctcaagcagtcttcctgcctcagcctctaaaattgctgggattacaggcttgagtcaccatgcccagcctgaagtagcatttctaccctgtttaataattcagcagcttgtcatgtaagatattcatatatgcatataaaacattaggcagcttaatttggtaaaactgtaaaatggaaattttaaattgtttgcagcatcaataacattgatgtcagtatgatttttacatgctgatcttgaccaatttgaaacagtgagttaaaatctggctgatccgtactaatcctaaagaaatattctatgaactattaaatgtttccagaatatataaagaaacattatgatgtcaacacacccatctatttttttttggaaataaaaactccatttttcttattaaagaaaacatgcttattagaaaacatacggctgggtgcagtggcacacatgtaattccagtgctttgggagatcgaggtgggagaatcacttgaggccaggagtttgagaccagcctagacaacataatgagaccccctctctacacaaaaagaattagttgtgcatggtggcgtgcacctgtagtcccagctacttgggaggcagaggcaggagcatcccttgagcctaggagtttgagactgcaggagttcgagactgagtggaatgcagtggaactgcattccagcctgagtgacagagggagaccctgtcttaaaaaaataagaaagaaaacacaactgcagaaaattataaaggatttaagtcattccaaatatcactgccactttttatttagaatattctaaagaattctctctctgtgtacacacacacatatgcgtactcttaatccaagtagcttggtaggattttatttacctagtgcctagatgggaaattgcctggggattccaaatacctatttcattaaattaaagatgtcactgattttaagacttaacactatttttcatactgccaagaaagaaaacactaccagttataaatgtaaattgccatcaattgtaatacatcaattttagagctattattaataaaatgtgaatgtgcatcttagagcaatgaaatatagtactatatatttgatgaccttttctgccctgtgatattcagaaagtgaaagttaaatatgggctgagcatggtggctcacacctgtaatcccagtactttgggaagtcaagacgggaggctggcttgaacccaggagttcaagaccagcctaggcaatgtagcgagacgccatctcaaaatattaaaaataagtaaataagtaaataaaaagaaggttaagtatacaaatgtatttcctttgttgtgaatttatttcaattttatagtgattttttttttttgagacgaagtctcactcttgtcccccaggctggagtgcgatggcgtgatctcagctcactgcaacctctgcctcccaggttcaagctatactcctgccttggccccccgagtagctgggattacaggcgcctgctaccatgcctggctaatttttgtatttttagttgagatggggtttcaccatgttggccaggctggtctagaactcttgacctctggtgatccacccgcctcggactcccaaaatgctgggattacaggcgtgagccaccgtgcctggccagtggttttttgttgttgttgttgttgttgttttgtttttgtttttgtttttgttttgagacaggatcttgctctgtcacccaggctggagtgcagtggtgccatcttggttcactgcaacctctgcgtgggctcaagcaatcctcccacctccctttccagagtagcggggaccacaggtgtgtgccaccacacctgactaatttttgcatttttttttgtagaaacagggttttgccatgttgcccaggttggtctgaaactcctgagctcaaacaatccaactgccttggcttccctaagtgaaattacaggcatgggccactgtacccagtctagtgatttttttatttttatttttattttattttattttatttttttaccaaaaaaacaacaaagcctcaggaggaaaagttgatacacaagtaaattttattggaaatgtttttgtgtggaccttaagcagagggaaaattagtctgcattatggtgtatccagactaaatgactgatattaaaatgaaattattcttaggatttgcaatcttagagaaaactttttcatttttatttttttgagttacaaattatcttcatttacatttgagaacagtgagtcacagagggattaagtaacttactcaagatcatacaagtctttgatttgaacccaatcttttaactctgcagaactcagagtcactcttatttggaaaaactttttaactgatgtggatcctctaatatgggcttcctattattcattctctattagtcagaagttttgcaagcagacagaattcattttgccaattacgggattttccctcagttgcagtcaaggttcataaaactataactctttatctttaattagaaatgtttttttttttgagacaaggtcttgctctgttgcccagactggaatgcagtggcatagtggcccattgcagctttgaactcctgggctcaagggatcctctgcctcagcctcccaagtatctgagactacaagtgcgtgccatcacccatggctattttaaaaaaaaaaaaaattgtagagatagggtcttgctgtgttgcccaggctggtctcaaactcctggtctcaagcaatccttctgccttggtctcccaaagtgctgagattacaggtgtcagccg

SEQ ID NO: 34 - DNA, 26 bp, Artificial Sequence
  Primer
aagcttagatcacagctcactgtggc

SEQ ID NO: 35 - DNA, 26 bp, Artificial Sequence
  Primer
actagtcggctgacacctgtaatctc

SEQ ID NO: 36 - DNA, 2033 bp, Artificial Sequence
  Gap element
caacctactgtggcatagactttaatcctctgggtattcttttgttgttctttcctggtatatgctgtggaattgagatagactggttcgtgagcgagagattttgtgttgccacaggtaggacatgctcaaacaatacttgggtcatttcttgacccaagtcatctattcaccatagttttgtagcaccgatcttgcatacatttcatgtatcttctttgaaccccacgtcagtgctgcttatatgatactcagaaattaaacactaaggaataagattttcaggtaggattgagttttggagggtcacaaatcttgtaatgtctaatatttccactctccctgctgagaattagttttggcttccttggaggtgatatcgcctctgttgagtataagtggcctactgtgatcacaccactgcactccagcctgggtgacagagtgagaccctgtctcagaaaaaaaaaaaaaaaaaaagaatgcatggcctagatgacttctaaggtttttcccacccagttccagttttcatgttctaggcagagcagtaaagtgagaaacacatggacttgggagtttagtctcgcatttcactgccacttaatctgagcgactattccatatttaatctctctgaatgtatttactcatctttaaaggggaatgattattaacatctttttctcagggaaactatatgagtcaaggagataatatatttgaaaatctttttaactgcaaagcgctgtttcactgttggttataatgtgattgatctcattgtagtgagcagctgcttaattgcgttttagaatgtagggaagatagtaatatttttcacattatatatgtagctggttctggaactgtaaacatactccttttttatggagatctgagtcacgtaccataaaattcactcttttaaagttgtacaatccagtggtttttgatatattcagagttgtgcatctgctaccactatttcattttggaacccaaagaaaccttgtacccattagcagtcattctcccttctcccagcccctggcaactactaatctactttctacagaaagtccgtacagatttgtgtattatggacattccatataaatggactcatgcaatatcctgtcttctttcacttagcatagtgttttcaaggttcatctaggttggggcatgtatcagtacttcatcccttgttttggctgaataatatttcattgtacaaatatatcacattttgcttatccatctgttggtgaacatttgagtttctacctgttggcttttatgaataatgttgatttgaatgtttgtgtacaagtatgaatacctgttttcaggtctcttgagtatatagttgctaggtcatatagtaactctgtgtttaacattttgaggaattgcccgactatttaacaaggtatatgtactgttttacaccagtaacatatgagggttccaatatctccacatccttgacaacacttgttactgtcctttttattgtagccatcctagtggctatgatgtggtatctcattgtggttttgatttgtgtttctctgatgctgatgatgttgaacatgttttcatctgcttattggccatttacatatatcttcttaagaacggttacccatttacagtatggaaaatgcttcagatgcaactctagtcatgccttagagatggagctttattaaacattcagatctctaggcatatgaagtgctgagttctcttgaactcctaatacagattgcactgagtttagtgataccttttctggagcattcctgagttcaggtagggagaagggtttttgctgtgattggcttgttatgttctttctaaatggaaatagaattgaagtgtctcctctctccatttattggaagagtcatgagggacataattagatgatcccttggagtctccggcttaggtcagtggttatctacttaggctgcacattggaatcacctgagagttaaaaaaccaggataacctctgcctgt

SEQ ID NO: 37 - DNA, 26 bp, Artificial Sequence
  Primer
acgcgtagccgaacctctcgcaagtc

SEQ ID NO: 38 - DNA, 30 bp, Artificial Sequence
  Primer
tttggcttttaggggtagttttcacgacac

SEQ ID NO: 39 - DNA, 58 bp, Artificial Sequence
  Primer
atgataacttcgtatagcatacattatacgaagttatccatggtgagcagggcgagga

SEQ ID NO: 40 - DNA, 60 bp, Artificial Sequence
  Primer
tctagataccgttcgtataggatactttatacgaagttattaccacatttgtagaggttt

SEQ ID NO: 41 - DNA, 55 bp, Artificial Sequence
  Primer
ggtaccgaagccgctagcgctaccggtcgccaccatgtccaatttactgccgtac

SEQ ID NO: 42 - DNA, 26 bp, Artificial Sequence
  Primer
agatctctaatcgccatcttccagca

SEQ ID NO: 43 - DNA, 20 bp, Artificial Sequence
  Primer
gacacatgcagacagaacag

SEQ ID NO: 44 - DNA, 20 bp, Artificial Sequence
  Primer
aacttctcggggactgtggg

SEQ ID NO: 45 - DNA, 20 bp, Artificial Sequence
  Primer
ctatcgccttcttgacgagt

SEQ ID NO: 46 - DNA, 20 bp, Artificial Sequence
  Primer
gtttgctaacaccccttctc

SEQ ID NO: 47 - DNA, 19 bp, Artificial Sequence
  Primer
gggagctcaaaatggagga

SEQ ID NO: 48 - DNA, 20 bp, Artificial Sequence
  Primer
gaagagttcttgcagctcgg

SEQ ID NO: 49 - DNA, 20 bp, Artificial Sequence
  Primer
gcttgcattgtatgtctggc

SEQ ID NO: 50 - DNA, 20 bp, Artificial Sequence
  Primer
ggttctttccgcctcagaag

SEQ ID NO: 51 - DNA, 20 bp, Artificial Sequence
  Primer
gggagctcaaaatggaggac

SEQ ID NO: 52 - DNA, 20 bp, Artificial Sequence
  Primer
gcaataggtcaggctctcgc

SEQ ID NO: 53 - DNA, 20 bp, Artificial Sequence
  Primer
gaatgggttggcggagctac

SEQ ID NO: 54 - DNA, 21 bp, Artificial Sequence
  Primer
gctgtggttctgattgtgtgg

SEQ ID NO: 55 - DNA, 20 bp, Artificial Sequence
  Primer
gggagctcaaaatggaggac

SEQ ID NO: 56 - DNA, 21 bp, Artificial Sequence
  Primer
gctgtggttctgattgtgtgg

SEQ ID NO: 57 - DNA, 20 bp, Artificial Sequence
  Primer
gactacaagtgcgtgccatc

SEQ ID NO: 58 - DNA, 21 bp, Artificial Sequence
  Primer
gctgtggttctgattgtgtgg

SEQ ID NO: 59 - DNA, 23 bp, Artificial Sequence
  Primer
acgaggaccctgaagtgaagttc

SEQ ID NO: 60 - DNA, 24 bp, Artificial Sequence
  Primer
tgttcctctctaggcttggtcttg

SEQ ID NO: 61 - DNA, 17 bp, Artificial Sequence
  Probe
tacgtggacggcgtgga

SEQ ID NO: 62 - DNA, 22 bp, Artificial Sequence
  Primer
tctactccgcctccttcctgta

SEQ ID NO: 63 - DNA, 22 bp, Artificial Sequence
  Primer
tggcagtagtaggtggcgaagt

SEQ ID NO: 64 - DNA, 15 bp, Artificial Sequence
  Probe
atccggcaccgactt

SEQ ID NO: 65 - DNA, 3097 bp, Artificial Sequence
  Gap element
aagcttgctcattgacactctgtacccattaatcctccttattagctccccttcaatggacacatgggtagtcagggtgcaggtctcagaactgtccttcaggttccaggtgatcaaccaagtgccttgtctgtagtgtcaactcattgctgccccttcctagtaatccccataatttagctctccatttcatagtctttccttgggtgtgttaaaagtgaccatggtacactcagcacggatgaaatgaaacagtgtttagaaacgtcagtcttctcttttgtaatgccctgtagtctctctgtatgttatatgtcacattttgtaattaacagcttgctggtgaaaaggaccccacgaagtgttggatataagccagactgtaagtgaattactttttttgtcaatcatttaaccatctttaacctaaaagagttttatgtgaaatggcttataattgcttagagaatatttgtagagaggcacatttgccagtattagatttaaaagtgatgttttctttatctaaatgatgaattatgattctttttagttgttggatttgaaattccagacaagtttgttgtaggatatgcccttgactataatgaatacttcagggatttgaatgtaagtaattgcttctttttctcactcatttttcaaaacacgcataaaaatttaggaaagagaattgttttctccttccagcacctcataatttgaacagactgatggttcccattagtcacataaagctgtagtctagtacagacgtccttagaactggaacctggccaggctagggtgacacttcttgttggctgaaatagttgaacagctttaatatacaataattgttgcattattatttcagatgataaatgtggtcataagtaagaaataaatgatcgagtttagtcttttaattcactgtcctttgaatacctgcctcttactctggaggcagaagtcccatggatgtgtttatgaacatggttgaggaagatttaggaagactgcaacagtacactacctaaagtcgcgatgaaagtgtgagttccggagcctgacaactgattcaaagcatggcttagtacttcctaactctgaccttgggcaagttacttaacctctctgtgtcccatatgtgattagggtgaggttgataatagcagccatagagttaagaggattaagtgctataatgcaagtagagctcttacaacagtttctggtaaatcactcaataaattcagacatactattattttaagaaatctcaaagagttttcttgtaccttaaaattctcctagtgtgaaccattggttttggtatattgtgcttccatgtagtttaatatcaagatgtttttagatttcccttttaatttatttgttgacccattggttgttcaggagcatgctgtttacctgaaaataatggagatattaaggtatttgaatatttatcttctagtacattgaaaaactttttgagagtaaccaataataaatgatggaatgctactgctttttttttttgaagctgccagttattgtttacttacactatgccaaatataaaggcattaatctcataaaagtttcacaacaatcctgtgagggagacgatatccccattttacaaatcaggaaattaagacttaataaggttaaaagacttgcccaaagtcacagaaccagtaagtggtagagcttgaatttgaatacagacctgactctaaagctcttttctttctttagattttagtgttcattgcttacttgaatgagtatctataagaaaactttaacatgtaaaacttctgtgaaattatcttgtcccatatcagggtcatgtcaaactaatgtcctcctcagcatctttggaaaacttcagaggagaaatgagctttgcccctcctgttcatttcatataccactgttagacctgtccttccctttcagcatgctttgtccatatttagaagctgttgaagccattacttgtctggtcagtttttagtgctggaatggacctagccttttaggccttctgagatttagtttgatctcgtctttcccacctaatggctctgttctactacatagatttgatctgaaacagttctctgtttctaaaataactttcttttcatgatagtcacagtaaagtacatttattatggaaaaatcaataagtataacgagtgaaagttatttcttggtggtaagattatgggattatttgaactttctgtttcattgtattttatttatttatttatttttgtgatggagtctcactctgctgcccaggctggagtgcagtagtacgatcttggctcactgcaacctccccttcccagttcaagtgattctcctgcctcagactcccaagtagctgggattacaggcgcacgccaccatgcctggctaatttttttatctttagtagagacagggtttcaccatgttgaccaggctgatctccaactcctgatctcaggtatccacctgcctcagcctcccaaagtaccgggattacgggtgtgagccaccctgcctggcctcattttgtcttttgggggtatttttgtgtgcagatatatatgtatataaatatttttccctcttttccccagttagtatttgagcagatgaactttggacccgaatacctgtattcaagtctctaataccacttcttggctattttcattttatcaaatggcctcttatcctcgtttttctcatttattaagtagagatgtaactacttgatataattcaaaaactcaataatggcattcttttgttttttagactctagtgtctgtactcgttgtaccatgctgggattcatttgaacaattgcatggcttttttagtgtattattaaatttgcagtttacttagaatttactgggacctcatacaaatgggaaaaaaacataactgtgttactcatttgctgtgtgcctttggattgaccctattttttgtattcattttctccccatgtcctgagttccactttgaataaaaagtaatttttttcctgcctgtaaaataggctaccaataggactagt

SEQ ID NO: 66 - DNA, 20 bp, Artificial Sequence
  Primer
tcccaaagtaccgggattac

SEQ ID NO: 67 - DNA, 541 bp, Artificial Sequence
  Gap element
aatctttgtcagcagttcccttttaaatgcaaatcaataaattcccaaaaatttaactgctttatgaattcaatttaaaaatccttaaaataagtcctgtctctttaaaaaaacctatgcatagttatcatttctctacaaattaacctagtttagttttctgttggttccattttccttgtttgttaagttttagtagctagtttaattgtaatctcaatgattatgtggtagaatgggttggcggagctacaaaaattcctagctacttcagagacattaaatttcagacacatggtacactttatattacattttactatgctaaaataacacggctttcttttggaattctgttcagtttttcagattgtaatctcagctacatctcaacagattgttctcagatatgtcctattaccttctttgtgtagatagtgctttattgactaagaacaatgacaacaacaccttttgttttctgggaataggagaaaagttttaagccaaaactcttaattgcttatctgctccacgtgag

SEQ ID NO: 68 - DNA, 26 bp, Artificial Sequence
  Primer
aagcttaatctttgtcagcagttccc

SEQ ID NO: 69 - DNA, 493 bp, Artificial Sequence
  Gap element
tcatctctcaacaactctggggtggcattattattcccacttttcagataaggttactgaggcatagggaattgtccaaaggtacagagctagtccgctatagagatgagatttgaacccagggaacctggctcacagtttatgcttttgctaccttaagtttttaatagagtgacatcaaacaaacatttaagaatatgtttttcttttccttttataatttcattaaaaacattaagtctctgatcagtctgcagtttttatgtaggggtcaggtaatgttctaacttctgctttttcctaagtgattaacaggtttttataagcccttttgaaaaaatcacggtatctgtcgagcatctttgaatcagagtaagccttctagtgagtcatatgtcagcagtttgactgtatgggcttttctaatatccagttcaagtgtttatcagtgagtttttcttttaaatagatttgggacaggtactatgagagt

SEQ ID NO: 70 - DNA, 502 bp, Artificial Sequence
  Primer
gggggtatttttgtgtgcagatatatatgtatataaatatttttccctcttttccccagttagtatttgagcagatgaactttggacccgaatacctgtattcaagtctctaataccacttcttggctattttcattttatcaaatggcctcttatcctcgtttttctcatttattaagtagagatgtaactacttgatataattcaaaaactcaataatggcattcttttgttttttagactctagtgtctgtactcgttgtaccatgctgggattcatttgaacaattgcatggcttttttagtgtattattaaatttgcagtttacttagaatttactgggacctcatacaaatgggaaaaaaacataactgtgttactcatttgctgtgtgcctttggattgaccctattttttgtattcattttctccccatgtcctgagttccactttgaataaaaagtaatttttttcctgcctgtaaaataggctaccaatagg

SEQ ID NO: 71 - DNA, 26 bp, Artificial Sequence
  Primer
aagcttgggggtatttttgtgtgcag